SEQUENCE LISTING (2 sequences)

SEQ ID NO: 1 - protein, 195 aa, Homo sapiens
MGKISSLPTQLFKCCFCDFLKVKMHTMSSSHLFYLALCLLTFTSSATAGPETLCGAELVDALQFVCGDRGFYFNKPTGYGSSSRRAPQTGIVDECCFRSCDLRRLEMYCAPLKPAKSARSVRAQRHTDMPKTQKYQPPSTNKNTKSQRRKGWPKTHPGGEQKEGTEASLQIRGKKKEQRREIGSRNAECRGKKGK

SEQ ID NO: 2 - protein, 153 aa, Homo sapiens
MGKISSLPTQLFKCCFCDFLKVKMHTMSSSHLFYLALCLLTFTSSATAGPETLCGAELVDALQFVCGDRGFYFNKPTGYGSSSRRAPQTGIVDECCFRSCDLRRLEMYCAPLKPAKSARSVRAQRHTDMPKTQKEVHLKNASRGSAGNKNYRM

(19) United States
(12) Patent Application Publication
(10) Pub. No.: US 2019/0330597 A1
(43) Pub. Date: Oct. 31, 2019

(54) COMPOSITION FOR CULTURE OF PLURIPOTENT STEM CELLS

(71) Applicant: The Francis Crick Institute Limited, London (GB)

(72) Inventors: Kathy Niakan, London (GB); Sissy Wamaitha, London (GB)

(21) Appl. No.: 16/476,796

(22) PCT Filed: Jan. 11, 2018
(86) PCT No.: PCT/GB2018/050066

§ 371 Date: Jul. 9, 2019

(30) Foreign Application Priority Data
Jan. 11, 2017 (GB) 1700504.2

(51) Int. Cl. C12N 5/074; C12N 5/073; C12N 5/0735

(52) U.S. Cl. CPC C12N 5/0696; C12N 5/0604; C12N 5/0606; C12N 2533/52; C12N 2501/16; C12N 2500/32; C12N 2501/105

(57) ABSTRACT

The present invention relates to a chemically defined or minimal cell medium for culturing pluripotent stem cells, including embryonic stem cells and induced pluripotent stem cells. The said medium may consist of the following components: a base medium, IGF1, a TGF-β family member and a glutamine supplement. The invention also relates to methods and uses involving the cell medium.

### FIELD OF THE INVENTION

The present invention relates to a chemically defined cell medium for culturing pluripotent stem cells, including embryonic stem cells and induced pluripotent stem cells. The method also relates to methods and uses involving the cell medium.

### BACKGROUND TO THE INVENTION

Human embryonic stem cells (hES cells) derived from epiblast cells of human preimplantation embryos recapitulate some aspects of their cell type of origin, and can also be maintained in culture indefinitely. The epiblast has the unique potential to form the embryo proper. Therefore, hES cells represent a useful tool for understanding how the epiblast is established and maintained during a transient period in the early stages of human development. Moreover, hES cells may also be a tool for clinical applications because of their potential to give rise to multiple different cell types if cultured under the respective appropriate conditions.

Additionally, induced pluripotent stem cells (iPS cells) can be generated by reprogramming non-pluripotent cells, such as skin cells (fibroblasts) to upregulate the expression of key genes identified as important for hES cell establishment or maintenance and downregulate genes associated with the previous differentiated cell type. The derivation process is usually carried out in human ES cell culture media, to select for successfully reprogrammed cells.

This reprogramming process reverts the fibroblasts into pluripotent-like cells that, similar to embryo-derived hES cells, have the potential to contribute to all the tissues that make up a fetus. This would, for example, potentially allow for personalised therapies by generating pluripotent cells from individuals. Therefore, iPS cell reprogramming aims to generate cells that are as close as possible, ideally identical to, human ES cells.

Human ES and iPS cells are capable of long-term proliferation in vitro, while retaining the potential to differentiate into all cell types of the body. Thus, these cells could potentially provide an unlimited supply of patient-specific functional cells for both drug development and therapeutic uses. In addition, hES and iPS cells, with their unlimited proliferation ability, have a unique advantage over somatic cells as the starting cell population for differentiation to clinically relevant cell types.

Both hES and iPS cells can be maintained in similar culture conditions, which are generally those previously determined to be suitable to derive hES cells from human embryos (as the aim is for iPS cells to be equivalent to hES cells). Classical media combinations tend to utilise animal-derived serum or otherwise undefined or variable components. This is problematic for clinical-grade culture systems, which should ideally be xeno-free and have defined ingredients for consistency and to minimise batch-to-batch variation. The majority of hES cell media to date contains exogenous fibroblast growth factor (FGF), often bFGF or FGF2, added either to the media or secreted by mitotically inactivated feeder (MEF) cells on which the cells may be cultured.

However, it is becoming increasingly apparent that existing hES cells (and by extension, iPS cells derived to be equivalent to these cells) are not completely identical to the epiblast within the human embryo from which they were derived. Thus, even though they may be technically pluripotent, they may exhibit some biases in terms of differentiation potential, have a transcriptional and epigenetic status that is distinct, and may not provide an exact model of how the human epiblast is established or maintained. This presents challenges in using hES cells as a model to explore the basic biology of this developmental stage and as a clinical source of cells to model and treat human diseases.

There is thus a need in the art for alternative media for hES and iPS cell culture that is based on, and therefore recapitulates, the signalling environment in the human embryo, and excludes any unnecessary and possibly detrimental factors. Moreover, there is a needed to identify culture conditions that are Good Manufacturing Practice (GMP) compliant and are thus chemically defined and xeno-free.

The present inventors investigated further the signalling requirements for the establishment and maintenance of the pluripotent epiblast in the developing human preimplantation embryo. The inventors used their informed molecular understanding of human epiblast cells to develop a defined hES and iPS cell culture media.

### SUMMARY OF THE INVENTION

The present inventors have surprisingly found that a medium which comprises insulin-like growth factor 1 (IGF1), but not fibroblast growth factor (FGF), can be used in the culture of pluripotent stem cells. As demonstrated in the present Examples, the addition of FGF to a human embryo in culture leads to a reduction in NANOG expression. NANOG is a pluripotency-associated transcription factor in embryonic stem cells and a decrease in NANOG expression therefore indicates a detrimental effect on general pluripotency gene expression. This was a surprising result as FGF is a standard component of hES and iPS cell culture media. Conversely, addition of IGF1 to the embryo culture medium leads to an increase in the proportion of NANOG-expressing cells within the inner cell mass (ICM).

The present invention therefore provides a cell medium which comprises IGF1, for example exogenous IGF1, and which is free or substantially free of FGF, such as exogenous FGF.

In one aspect of the invention the cell medium consists of only basal medium, IGF1, a TGF-β family member and a glutamine supplement. That is, the medium comprises only these components. No other components are present.

The present Examples show that both human embryonic stem cells and induced pluripotent cells can be maintained and derived in such a medium. Cells grown in a medium according to the invention showed the same morphological and molecular characteristics as cells grown in a commercially available medium for culturing pluripotent stem cells (mTeSR™1) or cells grown on a layer of mitotically inactivated fibroblasts (MEFs) in the presence of conventional media that includes basal media, L-glutamine, b-mercaptoethanol, knockout serum replacement plus the addition of exogenous FGF and optionally contains exogenous Activin. The present invention provides a chemically defined medium, i.e. a medium in which all of the chemical components are known, which is highly advantageous for the purposes of culturing pluripotent stem cells such as human embryonic stem cells and iPS cells as described herein. Chemically defined media are advantageous for reasons of regulatory compliance in relation to clinical safety and efficacy, for example.

The cell medium of the invention as described herein can be used in the culture of pluripotent stem cells, such as embryonic stem cells and induced pluripotent cells. The term “culture” as used herein is intended to encompass all aspects of in vitro culture, including for example derivation, establishment, maintenance and expansion of cells.

The term pluripotent stem cell as used herein is intended to mean a cell which is pluripotent. Pluripotent stem cells have the potential to differentiate into many different cell types, for example any of the three germ layers: endoderm, mesoderm and ectoderm.

In one aspect the pluripotent stem cell may be from a mouse, rabbit, cow, pig or non-human primate. In a preferred aspect the pluripotent stem cell is a human pluripotent stem cell.

In a preferred embodiment the pluripotent stem cell is an embryonic stem cell, most preferably a human embryonic stem cell.

As is known in the art, embryonic stem cells are pluripotent stem cells derived from early embryos. Embryonic stem cell lines (ES cell lines) are cultures of cells derived from the epiblast cells of the inner cell mass (ICM) of a blastocyst or earlier morula stage embryos. A blastocyst is an early stage embryo that is approximately five to 7 days old in humans and is composed of 100-300 cells. ES cells are pluripotent and give rise during development to all derivatives of the three primary germ layers: ectoderm, endoderm and mesoderm. In other words, they can develop into each of cell types of the adult body.

In an alternative embodiment the pluripotent stem cell is an induced pluripotent stem cell, most preferably a human induced pluripotent stem cell.

Induced pluripotent stem cells are a type of pluripotent stem cell artificially prepared from a non-pluripotent cell, typically an adult somatic cell, or terminally differentiated cell, such as a fibroblast, a hematopoietic cell, a myocyte, a neuron, an epidermal cell, or the like, by inserting certain genes or non-integrating mRNAs or chemicals, referred to as reprogramming factors.

Cells may be transduced, transfected, electroporated or nucleofected with any one or a combination of the transcription factors SOX2 (SRY-related HMG-box 2), OCT4 (Octamer-binding transcription factor 4), KLF4 (Kruppel-Like Factor 4), and c-MYC (V-myc avian myelocytomatosis viral oncogene homolog), L-MYC, N-MYC, NANOG, LIN28, SALL4, UTF1, TBX3, inhibitors of p53 and/or p21 and/or the presence of epigenetic modifying drugs such as 5′-azacytidine and RG108. One skilled in the art will appreciate that this list is not exhaustive, and is merely an example of some of the factors or combination of factors that have been used to generate induced pluripotent stem (iPS) cells resembling hES cells. These factors affect conversion of non-pluripotent cells into iPS cells. It is known in the art that adult mice can be derived from iPS cells. These reprogrammed cells acquire ES cell-like properties, and therefore have the potential to generate any tissue (Boland, et al. (2009) Nature 461:91-94; Quinlan, et al. (2011) Cell Stem Cell 9:366-373).

One skilled in the art would be aware of methods for culturing, isolating or producing embryonic stem cells.

For example, hES cells can be obtained from blastocysts, for example using methods as set out in Thomson, et al. (1995) Proc. Natl. Acad. Sci. USA 92:7844-7848; Thomson, et al. (1998) Science 282:1145; Thomson & Marshall (1998) Curr. Top. Dev. Biol. 38:133-165; Reubinoff, et al. (2000) Nat. Biotechnol. 18:399-404; Chen and Egli et al., Cell Stem Cell, 2009 Feb. 6; 4.

Established ES cell lines are also available. Various hES cell lines are known and conditions for their growth and propagation have been defined, for example, hES cell lines Shef6, H1, H7, H9, H13 and H14. Any ES cells or ES cell lines are suitable for use according to the present invention.

ES cells may be derived from a blastocyst, by culturing the inner cell mass of a blastocyst, or obtained from cultures of established cell lines. Thus, as used herein, the term “ES cells” can refer to inner cell mass cells of a blastocyst, ES cells obtained from cultures of cells from the inner cell mass, and ES cells obtained from cultures of ES cell lines.

iPS cells may be obtained by various methods. For example, see the method of Takahashi, et al. (2007) Cell 126(4):663-76). The iPS cells are morphologically similar to hES cells, and express various hES cell markers.

Human embryonic stem cells may be defined by the presence of several transcription factors and cell surface proteins. Suitable transcription factor markers include OCT4, NANOG, and SOX2, and suitable antigen markers include the glycolipids SSEA3 and SSEA4 and the keratan sulphate antigens TRA-1-60 and TRA-1-81.

iPS cells also have characteristic antigens that can be identified or confirmed by immunohistochemistry or flow cytometry using antibodies for SSEA-1, SSEA-3, SSEA-4, TRA-1-60 and TRA-1-81. Such methods are routine in the art.

Pluripotency of embryonic stem cells can be confirmed by spontaneous or directed differentiation in vitro or by injecting approximately 0.5-10×10⁶ cells into the rear leg muscles of 8-12 week old male SCID mice, generating teratomas that demonstrate at least one cell type of each of the three germ layers.

The present invention extends to any cell or population of cells obtained or obtainable by any of the methods or uses of the invention as described herein.

### LIST OF FIGURES

FIG. 1: FGF treatment of human embryos. (A) Schematic of FGF treatment conditions (B) Immunofluorescence analysis for NANOG (pluripotent epiblast marker) and GATA6 (primitive endoderm marker) with DAPI merge at E6-7 in an embryo cultured in standard media (a) or in embryos cultured in media supplemented with 1 μg/ml FGF2 and 1 μg/ml heparin from E2.5 (b, b′). (C) Immunofluorescence analysis for NANOG and GATA6 with DAPI merge at E6-7 in standard media (a′) or in media supplemented with 100 ng/ml FGF2 from E2.5 (c). (D) Immunofluorescence analysis for NANOG and SOX17 (primitive endoderm marker) with DAPI merge at E6-7 in an embryo cultured in media supplemented with 100 ng/ml FGF2 from E5 (d). (E) Quantification of NANOG- and SOX17-expressing cells in control or embryos treated with 100 ng/ml FGF2 from E5 to E6-7. Scale bar=100 μm.

FIG. 2: IGF treatment of human embryos. (A) Boxplots of reads per kilobase of million mapped reads (RPKM) values for insulin and IGF ligands and receptors in human blastocysts as determined by single cell RNA-sequencing analysis (Blakeley et al., 2015, Development, 142:3151-3165). The range of expression is shown for human epiblast (EPI), primitive endoderm (PE) or trophectoderm (TE) lineages. Boxes correspond to the first and third quartiles, horizontal lines to the median, whiskers extend to 1.5 times the interquartile range and dots were outliers (B) Schematic of IGF1 treatment conditions. (C) Immunofluorescence analysis for NANOG with DAPI merge at E6-7 in an embryo cultured in media supplemented with 17 nM IGF1 from E2.5. (D) Quantification of NANOG-expressing cells in control or IGF-treated embryos. One-tailed t-test. *P<0.05; ns., not significant. Scale bar=100 μm.

FIG. 3: Expression of transcripts for selected laminin and integrin subunits in the human blastocyst. (A) Boxplots of reads per kilobase of million mapped reads (RPKM) values for Laminin-511 (LAMA5, LAMB1, LAMC1) and integrin α6β1 subunits (ITGA6, ITGB1) as indicated in human blastocysts as determined by single cell RNA-sequencing analysis (Blakeley et al., 2015, Development, 142:3151-3165). The range of expression is shown for human epiblast (EPI), primitive endoderm (PE) or trophectoderm (TE) lineages. Boxes correspond to the first and third quartiles, horizontal line to the median, whiskers extend to 1.5 times the interquartile range and dots were outliers. (B) Representative phase-contrast images of H9 hES cells grown in mTeSR™1 media on Matrigel or laminins LN-511 (BioLamina) and iMatrix-511 (Takara). Scale bar=150 μm.

FIG. 4: Establishing a method to culture established hES cells in the presence of Activin and IGF1 (AI medium). (A) Representative phase-contrast images of H1 hES cells grown for two passages on Laminin-511 (iMatrix-511) in control mTeSR™1 media or in A-DMEM, FGF2 and Activin, IGF1 alone, or Activin and IGF1 (AI medium). n=2 biological and n=2 technical replicates. Scale bar=300 μm. (B) Representative images of H9 hES cells adapted to 17 nM IGF1 and 50 ng Activin 2 days after passage either manually, or with various dissociation reagents−salt-free PBS, Gentle Cell Dissociation Reagent, ReLeSR or 0.5 mM EDTA. n=2 or 3 technical replicates. Scale bar=150 μm. (C) Representative images of Shef6 cells initially dissociated to a single-cell level using a Rho-associated kinase (ROCK) inhibitor and their subsequent growth. n=2 biological and n=3 technical replicates. Scale bar=1000 μm.

FIG. 5: Cells cultured in AI medium with inhibitors of FGF receptors or downstream MEK/ERK signaling. (A) Representative phase-contrast images of hES cells culture in AI media treated with 100 nM PD173074 (PD17, FGF receptor inhibitor), 1 μM PD0325901 (PD13, MEK inhibitor) or 10 μM SB-431542 (SB, Activin/Nodal receptor inhibitor). Cells were exposed to inhibitors for 72 hours. n=4 technical replicates. Scale bar=100 μm. (B) Representative immunofluorescence analysis of hES cells cultured in AI medium supplemented with DMSO as a control or with an FGF receptor inhibitor, a MEK inhibitor or an Activin/Nodal receptor inhibitor. Expression of the pluripotency proteins OCT4 and NANOG are shown together with DAPI nuclear staining. Scale bar=100 μm.

FIG. 6: Characterizing hES and iPS cells cultured in AI medium. (A) Representative phase-contrast images of hES cells cultured in KSR+FGF on MEFs, mTeSR™1 on Laminin-511 or AI medium on Laminin-511. n=2 or 3 biological and n=3 technical replicates. Scale bar=300 μm for the top row of images and 100 μm for the bottom row. (B) Representative phase-contrast images independently validating the culture of hES cells (Shef6 cell line) or iPS cells (CiB10) in AI medium. Scale bar=400 μm. (C) Cumulative cell population doubling of hES or iPS cells. (D) Viability assay on hES or iPS cells cultured in AI medium. (E) The number of viable hES or iPS cells per cm2 across 4 days of culture in AI medium. (F) Representative immunofluorescence analysis of hES cells (Shef6) cultured in AI medium. OCT4 or NANOG, TRA-1-81 or SSEA4 and DAPI nuclear staining. The images are also representative of iPS cells analysed. n=3 biological replicated for hES cells and n=2 biological replicates for iPS cells. Scale bar=50 μm.

FIG. 7: Cells cultured in AI medium robustly express pluripotency proteins. (A) Representative flow cytometry analysis of hES and iPS cells cultured in AI medium. The expression of pluripotency proteins TRA-1-60, SSEA4, CD30, SSEA3, NANOG, OCT4 and SOX2 was quantified. n=3 technical replicates. (B) The percentage of pluripotency protein expression in iPS and hES cells following single-cell passaging using ROCK-inhibitor (left graph) or (C) clump passaging (right graph). (D) Representative G-banding patterns of hES and iPS cells which were 46, XX and karyotypically normal.

FIG. 8: De novo derivation of hES cells from human preimplantation embryos in AI medium. (A) The inner cell mass (ICM) of human day 6 preimplantation embryos was dissected and plated in AI medium in the presence of MEF-coated plates. Following the initial ICM outgrowth the cells were passaged onto Laminin-511-coated plates. Scale bar=150 μm B) Representative immunofluorescence analysis of newly established hES cells cultured in AI medium for the expression of the pluripotency proteins OCT4, NANOG, SOX2 and TRA-1-81 and DAPI nuclear staining. n=3 biological replicates. Scale bar=100 μm.

FIG. 9: De novo derivation of iPS cell lines in AI medium. (A) iPS cell-like colonies could be detected within 12 or 18 days following transient transduction with Sendi viruses driving the exogenous expression of OCT4, SOX2, cMYC and KLF4 (circles). iPS cell derivation was performed in KSR+FGF, TeSR™-E8 or AI medium. Scale bar=300 μm top row and 100 μm bottom row. (B) TRA-1-60 staining of colonies that emerged following 18 days of culture in the medium indicated. Scale bar=100 μm. (C) Established iPS cell lines derived using AI medium. n=2 technical replicates. Scale bar=100 μm.

FIG. 10: Differentiation potential of cells cultured in AI medium. (A) Immunofluorescence images confirming spontaneous differentiation of newly established hES cells in AI medium into all three germ layers: SOX17 (endoderm), TUJ1 (ectoderm) and DESMIN (mesoderm) in green, DAPI nuclear stain overlaid in blue. Representative of n=2 technical and n=3 biological replicates. Scale bar=100 μm. (B) Directed differentiation of H9 hES cells adapted to AI medium (P20) towards functionally mature hepatocytes. Representative phase-contrast images are shown throughout the time-course. Scale bar=150 μm top row and 100 μm bottom row. (C) A time-course immunofluorescence analysis of H9 cells adapted to AI medium throughout the hepatocyte differentiation process. Cells initially express the endoderm markers CXCR4 and SOX17, then upregulate FOXA2 as they differentiate into foregut endoderm cells and eventually express alpha-fetoprotein (AFP) and cytokeratin 18 (CK18) as mature hepatocytes. Scale bar=100 μm.

As discussed herein, the cell medium according to the present invention comprises insulin-like growth factor 1 (IGF1), for example exogenous IGF1 which has been added to the cell medium. In one embodiment the IGF1 is human IGF1.

The term “exogenous” as used herein is intended to mean introduced from or produced outside the cell medium, i.e. not synthesised or produced within the cell medium, for example by a cell within the medium (endogenous or naturally produced such as a natural gene product).

There are a number of splice variants of the IGF1 transcript (see for example Philippou et al. Mol med 2014; 20(1):202-214 for further information). The amino acid sequence of human IGF1-B (which is the canonical sequence) is given in SEQ ID NO:1 below:

```
                10 20 30 40
MGKISSLPTQ LFKCCFCDFL KVKMHTMSSS HLFYLALCLL
                50 60 70 80
TFTSSATAGP ETLCGAELVD ALQFVCGDRG FYFNKPTGYG
                90 100 110 120
SSSRRAPQTG IVDECCFRSC DLRRLEMYCA PLKPAKSARS
              130 140 150 160
VRAQRHTDMP KTQKYQPPST NKNTKSQRRK GWPKTHPGGE
              170 180 190
QKEGTEASLQ IRGKKKEQRR EIGSRNAECR GKKGK
```

The sequence of the IGF1-A form is given below in SEQ ID NO:2:

```
                10 20 30 40
MGKISSLPTQ LFKCCFCDFL KVKMHTMSSS HLFYLALCLL
                50 60 70 80
TFTSSATAGP ETLCGAELVD ALQFVCGDRG FYFNKPTGYG
                90 100 110 120
SSSRRAPQTG IVDECCFRSC DLRRLEMYCA PLKPAKSARS
              130 140 150
VRAQRHTDMP KTQKEVHLKN ASRGSAGNKN YRM
```

The splice variants of IGF1 essentially give rise to the same mature IGF1 protein. Recombinant IGF1 mature protein is commercially available, for example from R&D Systems (Minneapolis, USA) catalogue number 291-G1 (7.6 kDa mature protein). Such commercially available sources of IGF1 may be used according to the present invention.

It will be appreciated that any of the proteinaceous factors used in the medium of the present invention (e.g., the IGF1 or Activin as described herein) can be recombinantly expressed or biochemically synthesized. In addition, naturally occurring proteinaceous factors can be purified from biological samples (e.g., from human serum, cell cultures) using methods well known in the art. The factors may also be commercially available, as discussed herein.

Biochemical synthesis of the proteinaceous factors of the present can be performed using standard solid phase techniques. These methods include exclusive solid phase synthesis, partial solid phase synthesis methods, fragment condensation and classical solution synthesis.

Recombinant expression of the proteinaceous factors of the present can be generated using recombinant techniques that are known in the art.

References to IGF1 are intended to encompass fragments, variants, derivatives and homologs of IGF1 that have the same function as IGF1.

In one embodiment the IGF1 fragment, variant, derivative or homolog (with a homologous sequence) may have at least about 60, 65, 70, 75, 80, 85, 90, 91, 92, 93, 94, 95, 96, 97, 98, 99 or 100% sequence identity to SEQ ID NO:1 or SEQ ID NO:2 and the same function as IGF1, for example preferably with respect to function in cell culture. In one embodiment the fragment, variant, derivative or homolog may have at least about 98% sequence identity to SEQ ID NO:1 or SEQ ID NO:2 and the same function as IGF1 for example with respect to function in cell culture.

Sequence identity may be assessed by any convenient method. However, for determining the degree of sequence identity between sequences, computer programs that make multiple alignments of sequences are useful, for instance Clustal W (Thompson et al., (1994) Nucleic Acids Res., 22: 4673-4680). Programs that compare and align pairs of sequences, like ALIGN (Myers et al., (1988) CABIOS, 4: 1-17), FASTA (Pearson et al., (1988) PNAS, 85:2444-2448; Pearson (1990), Methods Enzymol., 183: 63-98) and gapped BLAST (Altschul et al., (1997) Nucleic Acids Res., 25: 3389-3402) are also useful for this purpose.

Furthermore, the Dali server at the European Bioinformatics Institute offers structure-based alignments of protein sequences (Holm (1993) J. Mol. Biol., 233: 123-38; Holm (1995) Trends Biochem. Sci., 20: 478-480; Holm (1998) Nucleic Acid Res., 26: 316-9).

Multiple sequence alignments and percent identity calculations may be determined using the standard BLAST parameters, (using sequences from all organisms available, matrix Blosum 62, gap costs: existence 11, extension 1).

Alternatively, the following program and parameters may be used: Program: Align Plus 4, version 4.10 (Sci Ed Central Clone Manager Professional Suite). DNA comparison: Global comparison, Standard Linear Scoring matrix, Mismatch penalty=2, Open gap penalty=4, Extend gap penalty=1. Amino acid comparison: Global comparison, BLOSUM 62 Scoring matrix.

The term “homologous” is intended to refer to the degree of sequence identity (see above) between sequences of two amino acid sequences, i.e. peptide or polypeptide sequences. “Homology” may be determined by comparing two sequences aligned under optimal conditions over the sequences to be compared. Such a sequence homology can be calculated by creating an alignment using, for example, the ClustalW algorithm. Commonly available sequence analysis software, more specifically, Vector NTI, GENETYX or other tools are provided by public databases. Thus included in the scope of the invention are variants of the stated or given sequences, as long as the variant retains the functional activity of the parent i.e. the variants are functionally equivalent, in other words they have or exhibit an activity of the parent, for example in cell culture. Such variants may comprise amino acid substitutions, additions or deletions of one or more amino acid compared to the parent sequence.

By a “variant” of the given amino acid sequence is intended to mean that the side chains of, for example, one or two of the amino acid residues may be altered (for example by replacing them with the side chain of another naturally occurring amino acid residue or some other side chain) such that the peptide retains the functional activity of the parent peptide from which it is derived.

Variants may involve the replacement of an amino acid residue by one or more of those selected from the residues of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine.

Such variants may arise from homologous substitution i.e. like-for-like/conservative substitution such as basic for basic, acidic for acidic, polar for polar etc. Non-homologous substitution may also occur i.e. from one class of residue to another or alternatively involving the inclusion of unnatural amino acids such as ornithine, diaminobutyric acid, norleucine, pyridylalanine, thienylalanine, naphthylalanine and phenylglycine.

A substitution may be a conservative substitution. As used herein, a “conservative substitution” refers to changing amino acid identity at a given position to replace with an amino acid of approximately equivalent size, charge and/or polarity. Examples of natural conservative substitutions of amino acids include the following 8 substitution groups (designated by the conventional one-letter code): (1) M, I, L, V; (2) F, Y, W; (3) K, R, (4) A, G; (5) S, T; (6) Q, N; (7) E, D; and (8) C, S. Also included are functionally equivalent derivatives in which one or more of the amino acids are chemically derivatised, e.g. substituted with a chemical group. Functionally equivalent derivatives may be modified chemically by reacting specific amino acids either before or after synthesis of the peptide. Examples are known in the art e.g. as described in R. Lundblad, Chemical Reagents for Protein Modification, 3rd ed. CRC Press, 2004 (Lundblad, 2004. Chemical modification of amino acids includes but is not limited to, modification by acylation, amidination, pyridoxylation of lysine, reductive alkylation, trinitrobenzylation of amino groups with 2,4,6-trinitrobenzene sulphonic acid (TNBS), amide modification of carboxyl groups and sulphydryl modification by performic acid oxidation of cysteine to cysteic acid, formation of mercurial derivatives, formation of mixed disulphides with other thiol compounds, reaction with maleimide, carboxymethylation with iodoacetic acid or iodoacetamide and carbamoylation with cyanate at alkaline pH, although without limitation thereto.

The cell medium according to the invention comprises IGF1, for example IGF1, that is the IGF1 has been added to the medium and has not been naturally produced by a cell in the medium.

The IGF1 may be present in the cell medium of the invention at a concentration of between about 0.1 nM and 50 nM. For example, the IGF1 may be present in the cell medium at a concentration of about 1.0 to 20 nM, e.g. 1.2 to 18 nM, 1.7 to 17 nM or 1.5 to 15 nM. For example, the concentration of IGF1 may be about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2.0, 3.0, 4.0, 5.0, 6.0, 7.0, 8.0, 9.0, 10.0, 11.0, 12.0, 13.0, 14.0, 15.0, 16.0, 17.0, 18.0, 19.0, 20.0, 21.0, 22.0, 23.0, 24.0, 25.0, 26.0, 27.0, 28.0, 29.0, 30.0, 31.0, 32.0, 33.0, 34.0, 35.0, 36.0, 37.0, 38.0, 39.0, 40.0, 41.0, 42.0, 43.0, 44.0, 45.0, 46.0, 47.0, 48.0, 49.0 or 50.0 nM. In one embodiment the IGF1 in the cell medium is at a concentration of about 1.7 or 17 nM. In one embodiment the concentration is 1.7 nM. In one embodiment the concentration is 17 nM. In one embodiment the cell medium of the present invention as described herein does not comprise exogenous IGF2. That is to say, the cell medium according to the present invention is free, or substantially free, from IGF2, including exogenous IGF2. In one embodiment exogenous IGF2 has not been added to the cell medium. The term “IGF2” as used herein is also intended to encompass any homologs, variants or derivatives as defined herein of IGF2.

The cell medium according to the present invention is free, or substantially free, from fibroblast growth factor (FGF). The medium may be free of exogenous FGF. That is to say that exogenous FGF has not been added to the cell medium of the present invention, and the medium does not comprise exogenous FGF.

“Substantially” is used herein in accordance with its plain and ordinary definition to mean to a great extent or degree. For example, substantially free of FGF means to a great extent free of FGF, free of FGF to a great degree. Should numerical accuracy be required, depending on context, “substantially,” as used herein means, at least, 70%, 75%, 80%, 85%, or 90% or more, for example 91, 92, 93, 94, 95, 96, 97, 98, 99% or 100.

The FGFs are a family of growth factors with members involved in angiogenesis, wound healing, embryonic development and various endocrine signalling pathways. The term “FGF” as used herein is intended to encompass any member of the FGF family, for example FGF1, 2, 3, 4, 5, 6, 7, 8, 9, 10, 11, 12, 13, 14, 15, 16, 17, 18, 19, 20, 21, 22 or 23. The medium according to the present invention may be free or substantially free from one or more FGF selected from FGF1, 2, 3, 4, 5, 6, 7, 8, 9, 10, 11, 12, 13, 14, 15, 16, 17, 18, 19, 20, 21, 22 and 23. In one embodiment the cell medium of the present invention is free or substantially free from all FGFs 1, 2, 3, 4, 5, 6, 7, 8, 9, 10, 11, 12, 13, 14, 15, 16, 17, 18, 19, 20, 21, 22 and 23, i.e. no exogenous FGF as described herein may be added to the cell medium according to the present invention.

The term “FGF” as used herein is also intended to encompass any homologs, variants or derivatives as defined herein of any FGFs.

In one aspect the medium is free from or substantially free from FGF2, for example exogenous FGF2 or any homologs, variants, derivatives as defined herein thereof. As such, in this aspect no exogenous FGF2 is added to the cell medium according to the present invention.

The medium according to the invention may also be free, or substantially free, from activators of any FGF receptor (FGFR). Fibroblast growth factor receptors consist of an extracellular ligand domain composed of three immunoglobulin-like domains, a single transmembrane helix domain, and an intracellular domain with tyrosine kinase activity. These receptors bind fibroblast growth factors. Alternate splicing of four fibroblast growth factor receptor genes facilitates the production of approximately 48 different isoforms of FGFR. These isoforms vary in their ligand-binding properties and kinase domains, however all share the common extracellular region composed of three immunoglobulin (Ig)-like domains (D1-D3), and thus belong to the immunoglobulin superfamily.

The three immunoglobin (Ig)-like domains—D1, D2, and D3—present a stretch of acidic amino acids (“the acid box”) between D1 and D2 which may participate in the regulation of FGF binding to the FGFR. Immunoglobulin-like domains D2 and D3 are sufficient for FGF binding.

The cell medium of the present invention may be free or substantially free from activators of FGFR1, FGFR2, FGFR3, FGFR4, FGFRL1 (FGF receptor-like 1) and/or FGFR6.

Reference to an “activator” is intended to mean a substance or molecule which leads to activation of an FGF receptor, and thus leads to FGF signal transduction. For example, a fragment of portion of a full length FGF ligand may activate an FGF receptor.

In one embodiment the cell medium of the invention as described herein is free or substantially free from activators of FGF receptor FGFR1 or FGFR2.

In one embodiment the cell medium of the invention comprises an inhibitor of an FGF receptor. Suitable inhibitors will be known to one skilled in the art, and are commercially available.

An FGF receptor inhibitor may be selected from PD173074, Ponotinib, BGJ398, Nintedanib, Dovitinib, PRN1371, PD-166866, BLU-554, SUN11602, S49076, NSC12, Erdafitinib, AZD4547, Danusertib, Brivanib, Dovitinib, MK-2461, Brivanib Alaninate (BMS-582664), SSR128129E, LY2874455, SU5402, Dovitinib Lactate, FIIN-2, CH5183284 and BLU9931. In one aspect the inhibitor may be PD173074.

In one embodiment the cell medium of the invention may additionally or alternatively comprise a MEK inhibitor. Suitable inhibitors will be known to one skilled in the art, and are commercially available.

A MEK inhibitor may be selected from PD0325901, Arctigenin, BIX 02189, 10Z-Hymenialdisine, PD184352, PD198306, PD334581, PD98059, SL327, U0124 and U0126. In one aspect the inhibitor may be PD0325901.

In one embodiment the cell medium of the invention may additionally or alternatively comprise an ERK inhibitor. Suitable inhibitors will be known to one skilled in the art, and are commercially available.

An ERK inhibitor may be selected from SCH772984, DEL-22379, VX-11e, LY3214996, ERK5-IN-1, XMD8-92, SC1, Ulixertinib, FR180204 and GDC-0994.

In one aspect the medium may comprise a TGF-β inhibitor, for example SB-431542.

The cell medium according to the invention as described herein may comprise a TGFβ family member protein.

The TGFβ signalling pathway is involved in many cellular processes in both the adult organism and the developing embryo including cell growth, cell differentiation, apoptosis, cellular homeostasis and other cellular functions.

TGFβ family members include Bone morphogenetic proteins (BMPs), Growth and differentiation factors (GDFs), Anti-millerian hormone (AMH), Activin, Nodal and TGFβ's. In one aspect the TGFβ family member may be Activin or Nodal.

In a preferred embodiment of the invention the cell medium comprises an Activin. For example, exogenous Activin such as Activin A, Activin AB and/or Activin B may be present in the cell medium. Suitable sources of Activin for use according to the invention are commercially available, for example from R&D Systems (cat no. 338-AC/CF) or Peprotech (cat no. 120-14).

In one aspect the medium may comprise more than one TGFβ family member protein, for example a combination of different TGFβ family member proteins as discussed herein.

In one aspect of the invention the cell medium is free or substantially free from comprise any BMPs, for example any exogenous BMPs.

The TGFβ family member may be present in the cell medium of the invention as a concentration of between 1 ng/ml to 1 mg/ml. For example, the Activin may be present in the cell medium at a concentration of about 0.1, 0.2, 0.3, 0.4, 0.5, 0.6, 0.7, 0.8, 0.9 or 1 mg/ml. The concentration of Activin may be about 1, 2, 3, 4, 5, 6, 7, 8, 9, 10, 15, 20, 25, 30, 35, 40, 45, 50, 55, 60, 65, 70, 75, 80, 85, 90, 95 or 100 ng/ml. The concentration of Activin may be between about 10 and about 50 ng/ml. In one embodiment the concentration of Activin is about 10 ng/ml. In one embodiment the concentration of Activin is about 50 ng/ml.

The glutamine supplement may be any suitable glutamine supplement, such as Glutamax™, or glutamine such as L-glutamine.

In one aspect the glutamine supplement is Glutamax™. Glutamax™ is commercially available, for example from Gibco (catalogue no. 35050-038). In one aspect of the invention the glutamine supplement is Glutamax™ from Gibco.

The glutamine supplement may be present in the cell medium at a concentration of between about 0.5 and 10 mM, for example 0.5, 0.6, 0.7, 0.8, 0.9, 1.0, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.5, 6.0, 6.5, 7.0, 7.5, 8.0, 8.5, 9.0, 9.5 or 10.0 mM. In one aspect the glutamine supplement may be present in the cell medium at a concentration of between about 1 mM and 3 mM. In one aspect the glutamine supplement may be present in the cell medium at a concentration of about 2 mM.

In one embodiment of the invention the cell medium comprises a BMP inhibitor.

Originally discovered by their ability to induce the formation of bone and cartilage, BMPs are now considered to be important in orchestrating tissue architecture throughout the body. Seven BMPs were discovered originally. Of these, six (BMP2 to BMP7) belong to the Transforming growth factor beta superfamily of proteins. BMP1 is a metalloprotease. Thirteen further BMPs have since been discovered, bringing the total to twenty. Any of the BMPs, or a combination of one or more BMPs, may be inhibited in the cell medium according to the present invention.

Any suitable BMP inhibitor, or combination of BMP inhibitor, may be added to the cell medium of the invention as described herein. BMP inhibitors are known in the art. Such inhibitors may include inhibitors of the BMP and also BMP receptors.

For example, BMP inhibitors include but are not limited to DMH1 (4-[6-(4-Isopropoxyphenyl)pyrazolo[1,5-a]pyrimidin-3-yl]quinolone, for example as available from Sigma-Aldrich catalogue number D8946), Dorsomorphin (6-[4-(2-Piperidin-1-ylethoxy)phenyl]-3-pyridin-4-ylpyrazolo[1,5-a]pyrimidine, for example as available from Sigma-Aldrich catalogue number P5499), K02288 (3-[(6-Amino-5-(3,4,5-trimethoxyphenyl)-3-pyridinyl]phenol, 3-[6-Amino-5-(3,4,5-trimethoxyphenyl)-3-pyridinyl]-phenol, for example as available from Sigma-Aldrich, catalogue number SML1307-K02288) and LDN-193189 (4-[6-[4-(1-Piperazinyl)phenyl]pyrazolo[1,5-a]pyrimidin-3-yl]-quinoline hydrochloride, for example as available from Sigma-Aldrich, catalogue number SML0559). In one embodiment the BMP inhibitor is DMH1.

The base medium for the cell medium of the invention as described herein can be any suitable medium for culturing pluripotent stem cells. Suitable media for pluripotent stem cells are commercially available and known in the art or may be prepared by methods known to one skilled in the art in this field of technology. Base media that may be used according to the invention as described herein include, but are not limited to, Dulbecco's Modified Eagle's Medium (DMEM), Ham's F10 medium, Ham's F12 medium, Advanced DMEM, Advanced DMEM/F12, minimal essential medium, DMEM/F-12, DMEM/F-15, Liebovitz L-15, RPMI 1640, Iscove's modified Dubelcco's media (IMDM), OPTI-MEM SFM (Invitrogen Inc.), N2B27, MEF-CM and defined basal ESC medium, ExVivo 10, ESGrow or a combination thereof. In one embodiment the medium is Advanced DMEM/F-12.

Advanced DMEM/F12 is commercially available, for example from ThermoFisher Scientific. The medium may, in one aspect, have the following composition:

| | | | |
|---|---|---|---|
| | Molecular | Concentration | |
| Components | Weight | (mg/L) | mM |

Amino Acids

| | | | |
|---|---|---|---|
| Glycine | 75.0 | 18.75 | 0.25 |
| L-Alanine | 89.0 | 4.45 | 0.049999997 |
| L-Arginine hydrochloride | 211.0 | 147.5 | 0.69905216 |
| L-Asparagine-H2O | 150.0 | 7.5 | 0.05 |
| L-Aspartic acid | 133.0 | 6.65 | 0.05 |
| L-Cysteine | 176.0 | 17.56 | 0.09977272 |
| hydrochloride-H2O | | | |
| L-Cystine 2HCl | 313.0 | 31.29 | 0.09996805 |
| L-Glutamic Acid | 147.0 | 7.35 | 0.05 |
| L-Histidine | 210.0 | 31.48 | 0.14990476 |
| hydrochloride-H2O | | | |
| L-Isoleucine | 131.0 | 54.47 | 0.41580153 |
| L-Leucine | 131.0 | 59.05 | 0.45076334 |
| L-Lysine hydrochloride | 183.0 | 91.25 | 0.4986339 |
| L-Methionine | 149.0 | 17.24 | 0.11570469 |
| L-Phenylalanine | 165.0 | 35.48 | 0.2150303 |
| L-Proline | 115.0 | 17.25 | 0.15 |
| L-Serine | 105.0 | 26.25 | 0.25 |
| L-Threonine | 119.0 | 53.45 | 0.44915968 |
| L-Tryptophan | 204.0 | 9.02 | 0.04421569 |
| L-Tyrosine disodium salt | 261.0 | 55.79 | 0.21375479 |
| dihydrate | | | |
| L-Valine | 117.0 | 52.85 | 0.4517094 |

Vitamins

| | | | |
|---|---|---|---|
| Ascorbic Acid phosphate | 289.54 | 2.5 | 0.008634386 |
| Biotin | 244.0 | 0.0035 | 1.4344263E−5 |
| Choline chloride | 140.0 | 8.98 | 0.06414285 |
| D-Calcium pantothenate | 477.0 | 2.24 | 0.0046960167 |
| Folic Acid | 441.0 | 2.65 | 0.0060090707 |
| Niacinamide | 122.0 | 2.02 | 0.016557377 |
| Pyridoxine hydrochloride | 206.0 | 2.0 | 0.009708738 |
| Riboflavin | 376.0 | 0.219 | 5.824468E−4 |
| Thiamine hydrochloride | 337.0 | 2.17 | 0.0064391694 |
| Vitamin B12 | 1355.0 | 0.68 | 5.0184503E−4 |
| i-Inositol | 180.0 | 12.6 | 0.07 |

Inorganic Salts

| | | | |
|---|---|---|---|
| Calcium Chloride (CaCl2) | 111.0 | 116.6 | 1.0504504 |
| (anhyd.) | | | |
| Cupric sulfate | 250.0 | 0.0013 | 5.2E−6 |
| (CuSO4—5H2O) | | | |
| Ferric Nitrate | 404.0 | 0.05 | 1.2376238E−4 |
| (Fe(NO3)3″9H2O) | | | |
| Ferric sulfate | 278.0 | 0.417 | 0.0015 |
| (FeSO4—7H2O) | | | |
| Magnesium Chloride | 95.0 | 28.64 | 0.30147368 |
| (anhydrous) | | | |
| Magnesium Sulfate | 120.0 | 48.84 | 0.407 |
| (MgSO4) (anhyd.) | | | |
| Potassium Chloride (KCl) | 75.0 | 311.8 | 4.1573334 |
| Sodium Bicarbonate | 84.0 | 2438.0 | 29.02381 |
| (NaHCO3) | | | |
| Sodium Chloride (NaCl) | 58.0 | 6995.5 | 120.61207 |
| Sodium Phosphate dibasic | 142.0 | 71.02 | 0.50014085 |
| (Na2HPO4) anhydrous | | | |
| Sodium Phosphate | 138.0 | 62.5 | 0.45289856 |
| monobasic | | | |
| (NaH2PO4—H2O) | | | |
| Zinc sulfate | 288.0 | 0.864 | 0.003 |
| (ZnSO4—7H2O) | | | |

Proteins

| | | |
|---|---|---|
| AlbuMAX ® II | 400.0 | Infinity |
| Human Transferrin (Holo) | 7.5 | Infinity |
| Insulin Recombinant Full | 10.0 | Infinity |
| Chain | | |

Reducing Agents

| | | | |
|---|---|---|---|
| Glutathione, monosodium | 307.0 | 1.0 | 0.0032573289 |

Trace Elements

| | | | |
|---|---|---|---|
| Ammonium Metavanadate | 116.98 | 3.0E−4 | 2.564541E−6 |
| Manganous Chloride | 198.0 | 5.0E−5 | 2.5252524E−7 |
| Sodium Selenite | 173.0 | 0.005 | 2.8901733E−5 |

Other Components

| | | | |
|---|---|---|---|
| D-Glucose (Dextrose) | 180.0 | 3151.0 | 17.505556 |
| Ethanolamine | 97.54 | 1.9 | 0.019479187 |
| Hypoxanthine Na | 159.0 | 2.39 | 0.015031448 |
| Linoleic Acid | 280.0 | 0.042 | 1.4999999E−4 |
| Lipoic Acid | 206.0 | 0.105 | 5.097087E−4 |
| Phenol Red | 376.4 | 8.1 | 0.021519661 |
| Putrescine 2HCl | 161.0 | 0.081 | 5.031056E−4 |
| Sodium Pyruvate | 110.0 | 110.0 | 1.0 |
| Thymidine | 242.0 | 0.365 | 0.0015082645 |

In one embodiment the base medium is Global® medium from LifeGlobal®. This medium is a bicarbonate-buffered medium comprising glucose, lactate, pyruvate and all 20 amino acids. In one aspect the base medium comprises Sodium Chloride, Sodium Pyruvate, L-Arginin, L-Threonine, Potassium Chloride, L-Alanine, L-Cystine, L-Tryptophan, Calcium Chloride, L-Asparagine, L-Histidine, L-Tyrosine, Potassium Phosphate, L-Aspartic Acid, L-Isoleucine, L-Valine, Magnesium Sulfate, L-Glutamic Acid, L-Leucine, Glycyl-L-Glutamine, Sodium Bicarbonate, Glycine, L-Lysine, EDTA, Glucose, L-Proline, L-Methionine, Phenol Red, Sodium Lactate, L-Serine, L-Phenylalanine, and Gentamicin Sulfate* (10 μg/ml).

In one embodiment the medium has not been conditioned or pre-treated with feeder cells, for example mouse embryonic fibroblasts (MEFs) or other feeder or support cells.

In a further embodiment the medium does not comprise serum, i.e. is serum-free. Thus, in one aspect no serum has been added to the cell medium of the present invention.

In one aspect of the invention the medium does not comprise an ErbB3 ligand. As used herein, “ErbB3 ligand” refers to a ligand that binds to ErbB3, which in turn dimerizes to ErbB2, thus activating the tyrosine kinase activity of the ErbB2 portion of the ErbB2/ErbB3 heterodimeric receptor.

Non-limiting examples of ErbB3 ligands include:

Neuregulin-1; splice variants and isoforms of Neuregulin-1, including but not limited to HRG-β, HRG-α, Neu Differentiation Factor (NDF), Acetylcholine Receptor-Inducing Activity (ARIA), Glial Growth Factor 2 (GGF2), and Sensory And Motor Neuron-Derived Factor (SMDF);

Neuregulin-2; splice variants and isoforms of Neuregulin-2, including but not limited to NRG2-β; Epiregulin; and Biregulin.

The ErbB3 ligand may be selected from the group consisting of Neuregulin-1, Heregulin-β (HRG-β), Heregulin-α (HRG-α), Neu differentiation factor (NDF), acetylcholine receptor-inducing activity (ARIA), glial growth factor 2 (GGF2), motor-neuron derived factor (SMDF), Neuregulin-2, Neuregulin-2β (NRG2-β), Epiregulin, Biregulin and variants and functional fragments thereof. In one aspect the medium according to the invention does not comprise Heregulin-β (HRG-β).

The base medium may comprise other nutrients or components that are required. For example, the medium may comprise amino acids, minerals, salts, ascorbic acid, glucose, glutamine, phenol red, antibiotics, β-mercaptoethanol, serum, serum-supplement proteins and/or lipids.

In one embodiment of the invention the medium is a chemically defined medium, i.e. a medium in which all of the chemical components are known. A defined medium should have known quantities of all ingredients and no yeast, animal or plant tissue should be present. A chemically defined medium (CDM) is thus a nutritive solution for culturing cells which contains only specified components, preferably components of known chemical structure. A CDM is devoid of undefined components or constituents which include undefined components, such as feeder cells, stromal cells, serum, and complex extracellular matrices, such as Matrigel™ The chemically defined medium may be humanised. A humanised chemically defined medium is devoid of components or supplements derived or isolated from non-human animals, such as Foetal Bovine Serum (FBS) and mouse feeder cells. Conditioned medium includes undefined components from cultured cells and is not chemically defined.

A CDM may comprise a chemically defined basal medium supplemented with a serum-free media supplement and/or one or more additional components, for example transferrin, 1-thioglycerol, defined lipids, L-glutamine or substitutes, such as GlutaMAX-1™, nicotinamide, dexamethasone, selenium, pyruvate, buffers, such as HEPES, sodium bicarbonate, glucose and antibiotics such as penicillin and streptomycin and optionally polyvinyl alcohol; insulin; polyvinyl alcohol and insulin; serum albumin; or serum albumin and insulin.

The medium may be a minimal medium, which contains only those elements that are essential for cell culture.

Suitable conditions for cell culture are known in the art. For example, cell cultures may be maintained in a CO₂ atmosphere, e.g., 0% to 12%, to maintain pH of the culture fluid, O₂ at 0% to 20%, incubated at 37° C. in a humid atmosphere and passaged to maintain a confluence below, e.g., 85%.

The cell medium according to the present invention may be used in conjunction with a basement membrane. Given that the basement membrane is the first extracellular matrix that is produced by the developing embryo, it has been identified as a factor for modulating stem cell behaviour, and basement membrane molecules may be utilised as a substratum in vitro.

Examples of basement membrane molecules that may be used according to the invention include vitronectin, fibronectin, various types of collagen, laminin, keratin, fibrin, fibrinogen, hyaluronic acid, heparin sulfate, chondroitin sulfate, agarose or gelatin.

A suitable basement membrane which is reflective of the embryo milieu may be used, with the aim of providing an optimum cell medium/basement membrane combination.

In a preferred embodiment the basement membrane comprises laminin, and more preferably does not comprise molecules other than laminin, thus the basement membrane may only contain laminin, i.e. consists of laminin.

The present Examples show de novo derivation of human embryonic stem cells and iPS cells lines grown in a medium according to the invention as described herein in combination with laminin. The cells appear morphologically similar to cells grown in commercially available media and on other substrates, but the present invention has the advantage that the medium is chemically defined minimal media. Laminin is also advantageous over other commercially available substrates, e.g. Matrigel, as it is more chemically defined and less subject to batch variation.

Laminin is a protein of the extracellular matrix. Laminins form a major component of the basal lamina (one of the layers of the basement membrane). Laminins form part of the structural scaffolding of tissues, and are secreted by cells and incorporated into the extracellular matrix. Laminins are heterotrimeric proteins that contain an α-chain, a β-chain, and a γ-chain. The laminin molecules are named according to their chain composition. Thus, laminin-511 contains α5, β1, and γ1 chains.

In one embodiment of the invention described herein, laminin 511 or 521 may be used. In one embodiment laminin 511 is used. Laminins are commercially available, for example, from BioLamina (recombinant Laminin-511, and Takara/Clontech iMatrix-511, recombinant Laminin-511 E8 fragment, Laminin-521).

One skilled in the art would be able to formulate a suitable basement membrane, for example as used in the manufacture's recommended methods for the laminins described above. (Also see Miyazaki et al. Biochem Biophys Res Commun. 2008; 375(1):27-32; Rodin et al. Nat Biotechnol. 2010; 28(6):611-5; and Miyazaki et al. Nat Commun. 2012; 3:1236.)

In one aspect of the invention the laminin may be added to the medium according to the invention, for example, for the culture of embryos as described herein. That is, the medium as described herein may additionally comprise laminin.

The cell medium of the invention may be used in a method for culturing a pluripotent stem cell as described herein.

As such, the invention provides a method for culturing a pluripotent stem cell comprising culturing said cell with a cell medium of the present invention as described herein.

Alternatively put, the invention provides use of a cell medium according to the invention as described herein in the culture of a pluripotent stem cell.

All aspects and preferred embodiments of the cell medium as described herein may be used in the methods and uses of the invention.

In a further aspect of the invention the cell medium as described herein may be used in the culture of an embryo. In a preferred embodiment the embryo is a human embryo.

The invention also therefore provides a medium for the culture of an embryo.

The invention also provides a method for culturing an embryo comprising culturing said embryo with the medium as described herein. The invention does not encompass embryos produced by such a method.

The medium may provide improved culture conditions for embryos which may lead to more successful embryo culture, for example increasing the proportion of embryos that develop successfully for the purposes of research or IVF treatment.

One skilled in the art would be aware of how to culture an embryo using standard practices. Suitable methods may be found in, for example, Wiemer et al., 2002, Reprod Biomed Online, 5:323-327 and Anderson et al., 2002, Reprod Biomed Online, 5:142-147.

In a still further aspect, the present invention provides a method for screening for factors which are essential for the culture of embryos.

Such a method may comprise adding a factor to a base cell culture medium and culturing an embryo in said medium and then analysing the effect on protein or gene expression of the factor. The effect on the embryo of adding the factor may be assessed by comparing embryos that have been cultured in the medium without the exogenous factor to determine whether the factor is essential and/or advantageous to the embryo.

As such, the invention provides a method of screening for factors that are essential for the culture of human embryos, wherein said method comprises the following steps:
1. Preparing a medium by adding a factor to be analysed;
2. Culturing a human embryo in said medium;
3. Comparing said embryo to an embryo that has been cultured in a medium without said factor or in medium that contains an inhibitor or activator of said factor; and
4. Determining whether said factor is essential for culture of said embryo.

Standard methods and techniques may be used to carry out the above method. In one aspect comparing said embryos and determining whether said factor is essential for culture may be performed by determining whether said factor alters the proportion of epiblast cells. For example, immunofluorescence staining may be carried out on control and treated embryos using markers associated with the pluripotent epiblast, as well as a DNA counterstain to mark cell nuclei. An automated software tool may be used to detect and segment nuclei and thus determine the number of cells in each embryo (MINS 1.3, http://katlab-tools.org/) (Lou et al., 2014 Stem Cell Reports 2:382-397). The number of epiblast cells may be calculated as a proportion of the total cells in the embryo, with treated embryos then compared to controls to determine any statistically significant changes in proportion.

The invention will now be further described by way of Examples, which are meant to serve to assist one of ordinary skill in the art in carrying out the invention and are not intended in any way to limit the scope of the invention.

### EXAMPLE 1

Materials and Methods

Generation of a Human Blastocyst Transcriptome Dataset

To identify putative distinct growth factors or signaling pathways, an RNA sequencing (RNA-seq) dataset recently generated from analyzing single-cells isolated from human embryos was used (Blakeley et al., 2015, Development, 142:3151-3165). Briefly, blastocyst stage human embryos (E6-E7) were laser microdissected to separate the majority of the mural trophectoderm from the inner cell mass (ICM) and polar trophectoderm (polar TE). The ICM and polar TE were incubated in 0.05% trypsin/EDTA (Invitrogen) for 5-10 minutes and single cells isolated using a 30-μm inner diameter blastomere biopsy pipette (Research Instruments). RNA was extracted from single cells and processed for cDNA synthesis using the SMARTer Ultra Low RNA Kit for Illumina Sequencing-HV (Clontech Laboratories). cDNA was sheared using a Covaris S2 with the modified settings 10% duty, intensity 5, burst cycle 200 for 2 min.

Libraries were prepared using the Clontech Low Input Library Prep Kit according to the manufacturer's instructions. Library quality was assessed with an Agilent 2100 BioAnalyser and concentration measured with a Qubit 2.0 Fluorometer (Life Technologies). Libraries were submitted for 50-bp paired-end sequencing using standard Illumina adapters on Illumina HiSeq 2500.

The quality of the RNA-seq data was evaluated using the FastQC tool. Reads were aligned to the human genome sequence hg19 using Tophat2 (Kim et al., 2013, Genome Biol, 14, R36) and samples with low percentage mapping (<50%) were excluded from subsequent analysis. The number of reads mapping uniquely to each gene was counted using the program htseq-count (Anders et al., 2015, Bioinformatics, 31, 166-169). The individual count files for each sample were normalized using both the RPKM (reads per kilobase of million mapped reads) function in the edgeR package (Robinson et al., 2010, Bioinformatics, 26, 139-140). Additional human single-cell RNA-seq data (Yan et al., 2013, Nature Structural and Molecular Biology 20:1131-1139) was normalized using the RPKM method and integrated with our own blastocyst sequencing data.

To investigate differences in global gene expression, a PCA of the top 8000 genes with the most variable expression was performed on the human blastocyst samples using the R package prcomp. Cells were placed into three lineages: epiblast (EPI), primitive endoderm (PE) or trophectoderm (TE) and boxplots of RPKM values were generated to show the range of gene expression.

Extended methods are provided in Blakeley et al., 2015, Development, 142:3151-3165 (see Supplementary Materials and Methods).

Human Embryo Culture

Human embryos were donated to the research project by informed consent under the UK Human Fertilisation and Authority Licence number R0162. Vitrified embryos frozen in straws were thawed by quickly transferring the contents of the straw from liquid nitrogen directly into thaw solution (Irvine Scientific Vitrification Thaw Kit) and thawed as per manufacturer's instructions. Embryos frozen in cryopets were first thawed for 3 seconds in a 37° C. waterbath before transferring into thaw solution (Irvine Scientific Vitrification Thaw Kit). Embryos frozen in glass ampoules were thawed completely in a 37° C. waterbath after the top of the vial was removed under liquid nitrogen. The contents were emptied onto a petri dish and the embryo transferred through a gradient of sucrose solutions (Quinn's Advantage Thaw Kit, Origio) as per manufacturer's instructions.

Embryos were routinely cultured in Global Media (LifeGlobal) supplemented with 5 mg/mL LifeGlobal Protein Supplement pre-equilibrated in an incubator at 37° C. and 5% CO2. For growth factor or inhibitor treatment, these conditions were further supplemented with FGF2 (3718-FB-01M, R&D) or IGF1 (291-G1-10, R&D) as indicated.

Immunofluorescence

Embryos were fixed with 4% (w/v) paraformaldehyde (PFA) (ThermoFisher Scientific) for 1 h at 4° C. on a rotating shaker and analysed as described previously (Niakan and Eggan, Developmental Biology, 2013: doi: 10.1016/j.ydbio.2012.12.008). Briefly, embryos were then transferred through several washes of 0.1% (v/v) Tween-20 (Sigma Aldrich) diluted in Dulbeco's Phosphate-Buffer saline (PBS) without calcium and magnesium (Thermo Fisher Scientific) to remove residual paraformaldehyde. Embryos were placed for 20 minutes in 0.5% (v/v) Tween-20 for permeabilization. Embryos were blocked for 1 hour at room temperature in blocking solution (10% donkey serum diluted in 0.1% (v/v) Tween-20). Embryos were placed in primary antibodies at a concentration of 1:500 in blocking solution overnight at 4° C. on a rotating shaker. The following primary antibodies were used (all at 1:500 dilution): anti-NANOG (AF1997 R&D Systems, REC-RCAB0001P 2B Scientific, or ab21624, Abcam), anti-GATA6 (SC-9055, Santa Cruz) and anti-SOX17 (AF1924, R & D Systems). The following day, embryos were transferred through 4 washes of 0.1% (v/v) Tween-20 then placed in a last wash for 30 minutes. Secondary antibodies (Cy3, FITC or Cy5 donkey anti-rabbit, mouse or goat, Molecular Probes) were diluted in blocking solution at a 1:300 concentration. Embryos were placed in secondary antibody for 1 hour at room temperature on a rotating shaker, transferred through 4 washes of 0.1% (v/v) Tween-20 and placed in a last wash for 30 minutes. Embryos were placed in a 50 μL 1:3 dilution of Vectashield containing DAPI (Vector Labs): 0.1% (v/v) Tween-20 on a coverslip bottom dish (MatTek) for confocal imaging.

Confocal Imaging and Quantification of Immunofluorescence

Embryos were imaged on a Leica SP5 inverted confocal microscope (Leica Microsystems GmbH) at a z-section thickness of 3 μm or 2 μm for human or mouse embryos respectively. MINS 1.3 software was used to detect and segment nuclei and thus determine the number of cells in each embryo (http://katlab-tools.org/) (Lou et al., Stem Cell Reports 2014, doi: 10.1016/j.stemcr.2014.01.010). Confocal stacks in .tif format were loaded into the MINS pipeline for automated nuclear segmentation.

The MINS segmentation output was manually checked for appropriate segmentation and mitotic nuclei were removed from the analysis. Data were subsequently plotted using GraphPad Prism version 6 (GraphPad Software, La Jolla, Calif.).

Results

Investigation of the Effects of FGF on Embryo Culture

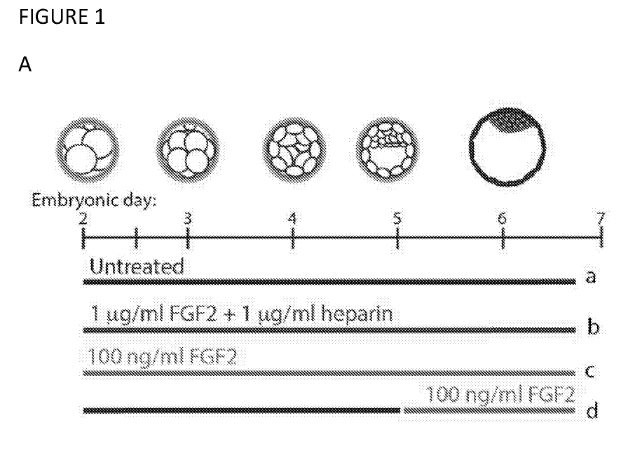

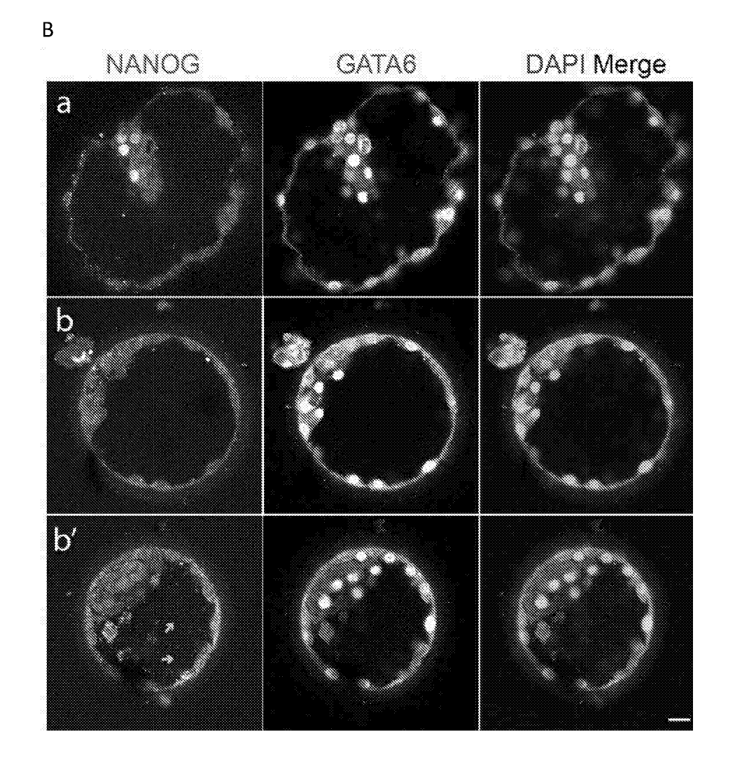

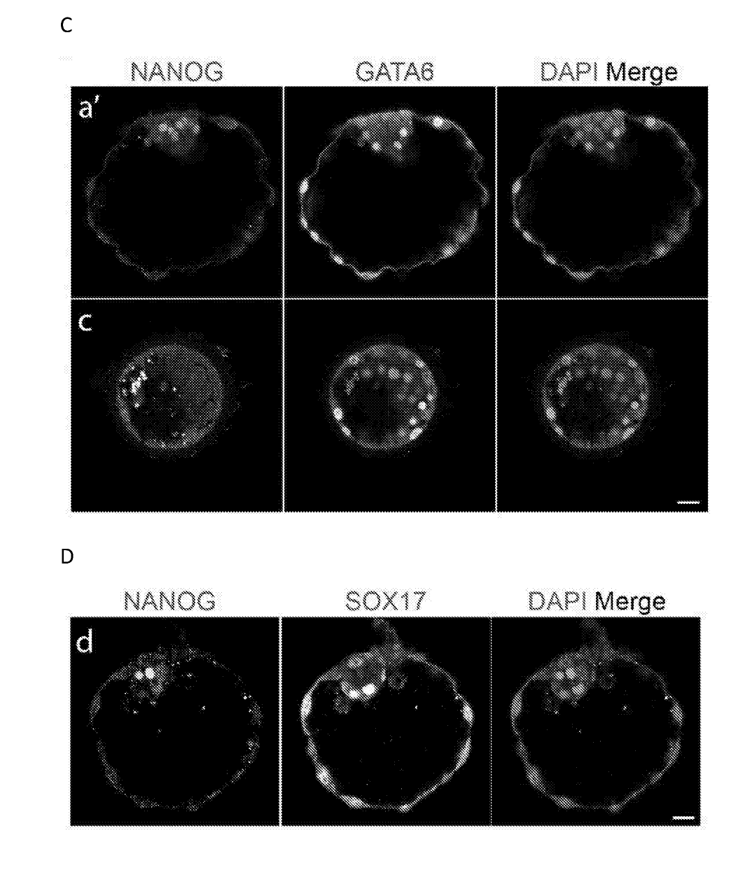

Human embryos were cultured from E2.5 or E5 in human embryo culture media supplemented with FGF2 at concentrations of 100 ng/ml or 1 μg/ml (FIG. 1A). Immunofluorescence analysis showed that when treated from E2.5 with either 100 ng/ml or 1 μg/ml, there was a loss of NANOG expression compared to untreated controls (FIG. 1).

This indicated that FGF addition had a detrimental effect on pluripotency gene expression in the human embryo (FIG. 1A-C).

In contrast, the extraembryonic marker GATA6 was still expressed in both instances, suggesting that the effect of FGF is specific to the pluripotent compartment. However, when treated with 100 ng/ml from E5, NANOG remained expressed (as did the extraembryonic marker SOX17) (FIGS. 1D and 1E), suggesting that shorter treatments at a low concentration may not be detrimental to pluripotency and equally does not affect extraembryonic lineages.

Investigation of the Effects of IGF1 on Human Embryo Culture

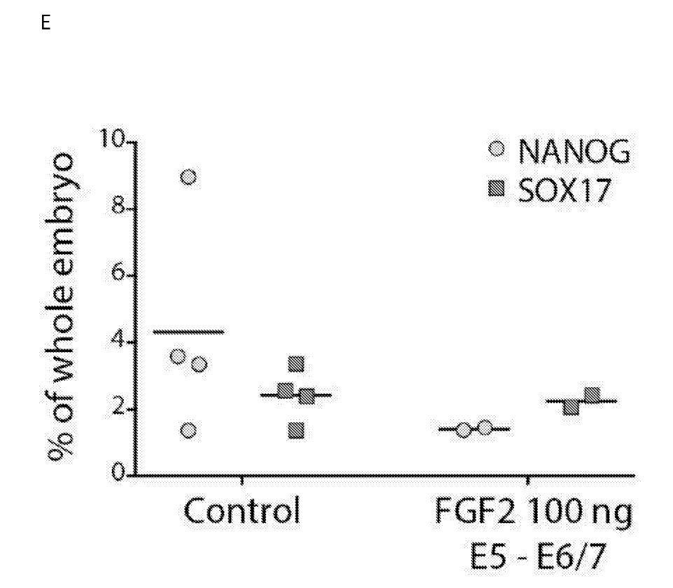

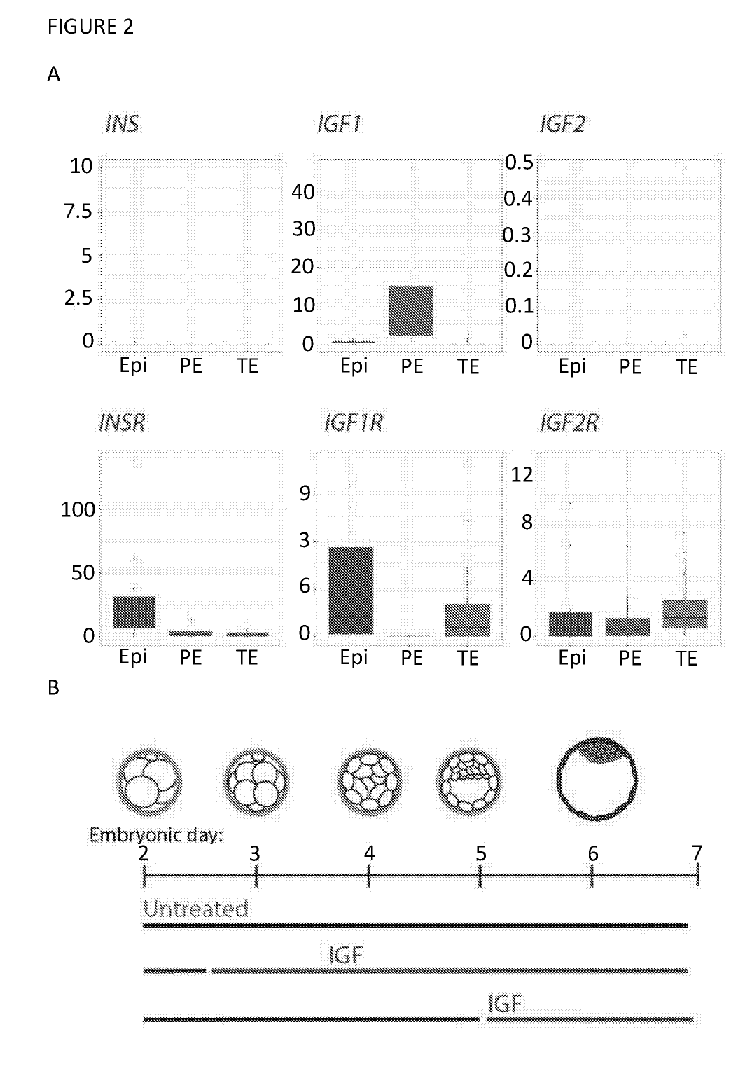

The transcriptome data set (produced as described above in Materials and methods) was interrogated to identify transcripts encoding receptors that are expressed in pluripotent epiblast cells, or encoding ligands expressed by any cell type in the human embryo. We identified expression of transcripts for the insulin (INSR) and IGF1 (IGF1R) receptors, along with IGF1 ligand (IGF1) in early human development (FIG. 2A). Notably, the expression of Insulin and IGF2 was undetectable in human blastocysts. The presence of transcripts for IGF1 and its cognate receptors in the human blastocyst indicated this might be an interesting candidate to modulate.

Human embryos were cultured from E2.5 in human embryo culture media supplemented with 17 nM IGF1 (FIG. 2B). At E6-7 embryos were analyzed for the expression of NANOG (FIG. 2C) via immunofluorescence. Automated quantification analysis showed that IGF1 treatment resulted in a 2-fold increase (p-value<0.05) in the proportion of NANOG expressing cells compared to control embryos (FIG. 2D). Moreover, the transcription factor KLF17, a marker enriched in human epiblast cells, was also expressed in a higher proportion of cells in human embryos treated with IGF1 (FIG. 2E). Altogether this showed a positive effect on pluripotency gene expression in human embryos exposed to exogenous IGF1 and suggested that IGF1 could be used during hES and iPS cell derivation.

Identifying an Appropriate Basement Membrane Substrate for hES Cell Derivation

Human ES cell derivation and culture systems often rely on a supportive MEF layer for cell propagation, but as MEFs secrete FGF, this was incompatible with our requirements. Supporting matrix substrate layers such as Matrigel, laminin and vitronectin are commonly used in place of MEFs in feeder-free culture systems. However, Matrigel is distilled from Engelbreth-Holm-Swarm (EHS) sarcoma cells and thus remains subject to batch-to-batch variability, and even in its growth factor-reduced form retains noticeable concentrations of various growth factors, including FGF. It was also unclear whether additional basement membrane substrates would reflect those present in the human embryo.

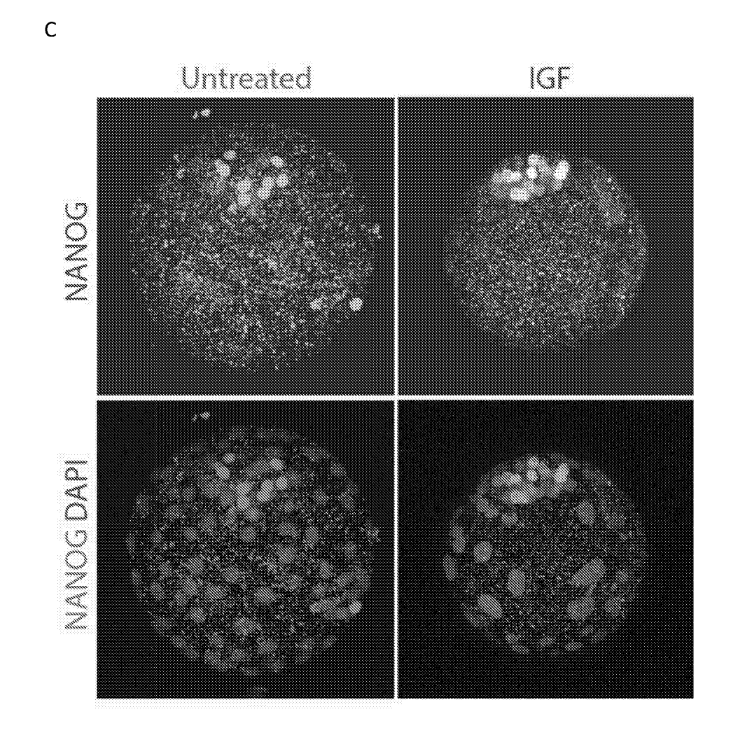

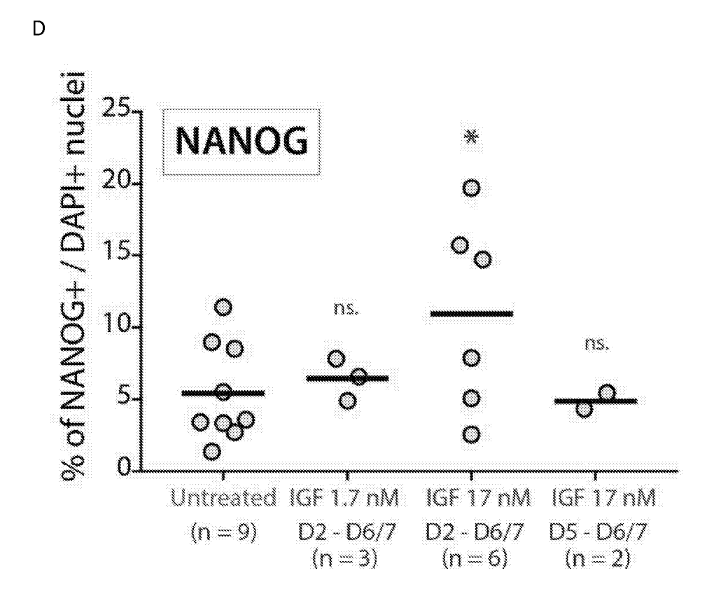

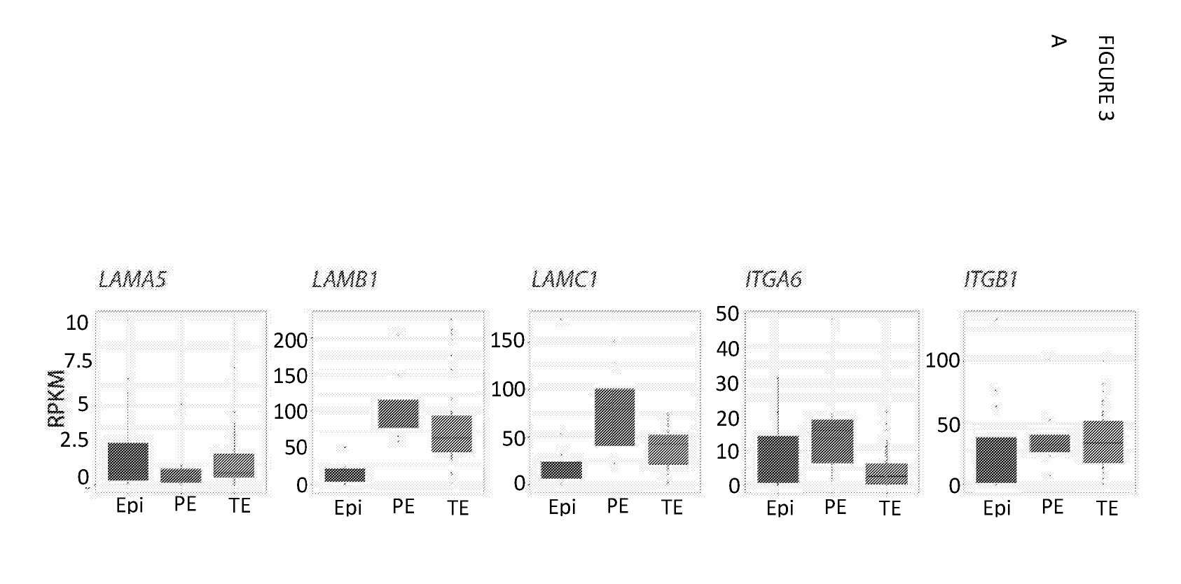

We therefore interrogated the transcriptome data set to identify chemically defined and physiologically relevant substrates for the derivation of hES and iPS cells. We also interrogated the transcriptome data set and identified expression of transcripts for laminin-binding Integrin α6 and Integrin-β1 (ITGA6, ITGB1) in the human blastocyst, as shown in FIG. 3A. We identified subunit transcripts for Laminin-511 (LAMA5, LAMB1, LAMC1), which Integrin-α6 and Integrin-β1 has previously been shown to bind with a high affinity (Nishiuchi et al., 2006, Matrix Biology, 25, 189-197). Consequently, recombinant human Laminin-511 (Biolamina) or a recombinant Laminin-511 E8 fragment (iMatrix-511, Takara) were selected as basement membrane substrates to culture hES cells, in order to recapitulate the niche within the blastocyst. To test the laminin variants, H9 cells in mTeSR™1 media were passaged onto plates coated with 0.5 μg/cm² of either iMatrix-511 (Takara) or LN-511 (Biolamina), as well as a control Matrigel plate, and cultured for 3 days. We determined that H9 cells could retain pluripotency morphology on iMatrix-511 or Laminin-511 equivalent to Matrigel coating (FIG. 3B).

### EXAMPLE 2

Materials and Methods

Culture Conditions for hES and iPS Cells

For culture in knockout serum replacement (KOSR) media supplemented with FGF2 (KSR+FGF), cells were maintained on MEF-coated pre-gelatinized tissue culture plates (Corning) in 20% KOSR and 5 ng/ml of FGF. Cells were passaged by manual picking.

For culture in mTeSR™1 media (StemCell Technologies), cells were generally maintained on Matrigel-coated (BD Biosciences) tissue culture plates. Matrigel coating was performed for one hour at room temperature (RT) as per the manufacturer's instructions Cells were passaged as clumps using ReLeSR (Stemcell Technologies). ReLeSR was added to wells for 30 seconds at RT then aspirated, then plates were incubated for 5 minutes at 37° C., quenched with mTeSR™1 and lightly tapped to dislodge clumps of the desired size. H1 and H9 cells were also adapted to plates coated with 0.5 μg/cm² Laminin-511 (Biolamina, Takara). Laminin coating was performed either overnight at 4° C. or for one hour at 37° C. as per the manufacturer's instructions. Cells on Laminin-511 were also passaged with ReLeSR, but with a 7-minute incubation at 37° C.

For culture in TeSR™-E8 media (StemCell Technologies), cells were maintained on vitronectin-coated (StemCell Technologies) tissue culture plates. Vitronectin coating was performed for two hours at RT as per the manufacturer's instructions. Cells were passaged as clumps using 0.5 mM EDTA. EDTA was added to wells for 5 mins at RT then aspirated, then TeSR™-E8 added to wells and cells disaggregated with a 5 ml stripette.

For culture in AI media, cells were maintained on plates coated with 0.5 μg/cm² Laminin-511 (Biolamina, Takara), and generally passaged with ReLeSR, similar to cells in mTeSR™1 on Laminin. Early passage newly-derived hES cells in AI medium were passaged by manual picking. Cells in AI media were also split using various dissociation reagents, as indicated in the Examples. For Accutase or AccuMax (StemCell Technologies), the dissociation reagent was added to wells for 5-10 minutes, the well contents were collected and pelleted, and the dissociation reagent removed. Cells were then resuspended in AI media with or without 10 mM ROCK inhibitor for plating. For Gentle Cell Dissociation Reagent (StemCell Technologies), the reagent was added to wells for 6 minutes, then aspirated and AI medium added. Well contents were collected and disaggregated with a 5 ml stripette. For 0.5 mM EDTA (Sigma), the reagent was added for 5 minutes, then aspirated, and AI medium added and cells disaggregated. For salt-free PBS (Gibco, Life Technologies), the reagent was added for 6 minutes, aspirated, then AI medium added and cells disaggregated.

For inhibitor treatment experiments, cell culture media was supplemented with 100 nM PD173074 (FGF receptor inhibitor), 1 μM PD0325901 (MEK inhibitor) or 10 μM SB-431542 (Activin/Nodal receptor inhibitor) as indicated in the Examples. Cells were exposed to inhibitors for 72 hours.

Immunofluorescence

On the day of fixation, medium was removed from pluripotent cell lines or differentiated cells and the cells were washed with Dulbeco's Phosphate-Buffer saline (PBS) without calcium and magnesium (Thermo Fisher Scientific). Cells were fixed with 4% (w/v) paraformaldehyde (PFA) (ThermoFisher Scientific) for 1 h and and permeabilised with 0.5% (w/v) Triton X-100 or 0.5% (v/v) Tween-20 (Sigma Aldrich). Non-specific binding was minimised using 2-10% (v/v) donkey serum (AbCam) in PBS (containing 0.05% (v/v) Tween-20 or 0.01% Tween-20). The following primary antibodies were used (all at 1:500 dilution unless otherwise indicated): anti-NANOG (AF1997 R&D Systems; REC-RCAB0001P, 2B Scientific; 4903P, Cell Signaling Technologies or ab21624, Abcam), anti-SOX17 (AF1924, R&D Systems), anti-TRA-1-81 (MAB4381, Millipore; or 560072, BD Biosciences), anti-TUJ1 (T2200, Sigma), anti-FOXA2 (3143, Cell Signaling), anti-OCT4 (sc-5279, Santa Cruz or 2750S, Cell Signaling Technologies), anti-SSEA4 (MA1-021, Life Technologies or MC-813-70, DSHB, 1:100), anti-TRA-1-60 (MAB4360, Millipore, 1:100), anti-CXCR4 (MAB173, R&D), anti-Desmin (RB-9014-R7, Neomarkers, 1:50), anti-AFP (A0008, Dako), anti-cytokeratin-18 (ab668, Abcam). Primary antibody was removed by washing with PBS (containing 0.05% or 0.01% v/v Tween-20). Secondary antibodies (Alexa Fluor® 488, Alexa Fluor® 594, Alexa Fluor® 555 donkey anti-rabbit, mouse or goat, Molecular Probes) were diluted in blocking solution at a 1:300 concentration and added to the corresponding wells for 1 hour at room temperature. Cells were the washed several times in of 0.1% (v/v) Tween-20 and placed in a last wash for 30 minutes. A drop of Vectashield containing DAPI (Vector Labs) was added to each well prior to imaging.

Imaging

Images were acquired using a Leica TCS SP8 confocal microscope or on an Olympus 1X73 microscope with Cell\hat{ }F software (Olympus Corporation). Phase contrast images were taken on a Leica DM IL LED microscope with a Leica MC170 H9 camera (Leica Microsystems GmbH).

Human ES Cell Derivation from Preimplantation Embryos

E5 or E6 human blastocysts were initially cultured in Global Media (LifeGlobal) supplemented with 5 mg/mL LifeGlobal Protein Supplement pre-equilibrated in an incubator at 37° C. and 5% CO2 prior to stem cell derivation. E6 blastocyst stage human embryos were disaggregated to isolate the inner cell mass using an Olympus IX73 microscope and a Saturn 5 laser (Research Instruments) as described in Chen et al., 2009, Cell Stem Cell: doi: 10.1016/j.stem.2008.12.001. Embryos were placed in drops of Global® Total® w/HEPES (LGTH, LifeGlobal) on a Petri dish overlaid with mineral oil for micromanipulation. The inner cell mass (ICM) and polar trophectoderm were plated onto MEF-coated dishes in AI medium. ICM outgrowths with hES cell-like morphology were manually picked onto either MEF-coated or Laminin-511 dishes for further propagation.

Human iPS Cell Derivation

Human BJ fibroblast cells were plated so as to be 30-60% confluent for transduction two days later (1×10⁵ cells per well of a 6-well). Cells were then transduced using the Cytotune™ 2.0 Sendai reprogramming kit (Invitrogen) according to the manufacturer's instructions, and transferred to either MEF-coated (KSR+FGF media), Vitronectin-coated dishes (TeSR™-E8 media) or Laminin-511 coated (AI media) dishes, 6 days after transduction. Cells were transferred to hypoxia conditions (5% O₂, 5% CO₂, 37° C.) at this point, and cultured under hypoxia for the remainder of the derivation. 18 days following transduction, TRA-1-60 expression was analyzed using the Stemgent® StainAlive™ TRA-1-60 Antibody (DyLight™ 488) kit, according to the manufacturer's instructions (1:100 dilution). Colonies with pluripotent ES cell morphology were picked 22 days after transduction for expansion to establish a stable iPS cell line.

Cell Growth and Viability

Cells from three random vials of a working cell banks were thawed in the ThawStar® device and cultured separately in the 2D culture system LN511 with AI media. Cell counts and viability were measured by Nucleocounter NC-200 (Chemometech) and population doublings calculated according to Equation 1.

$$PDL = \frac{{log}_{10} \frac{Viable cell number at time t}{Viable cell number at time t - 1}}{0.30103}$$

Flow Cytometry

Expression of surface- and intracellular-pluripotency related proteins was analysed by flow cytometry using a MACSQuant® Analyzer10 cytometer (Myltenyi Biotec). Single cell suspensions were washed and stained in BD Pharmigen™ Stain Buffer (BD Biosciences) with the following conjugated antibodies: SSEA4 (130-098-341, Myltenyi); SSEA3 (330306, Biolegend); CD30 (550041, BD Biosciences); TRA-1-60 (560495, Biolegend); NANOG (560791, BD Biosciences); OCT4 (561556, BD Biosciences); SOX2 (656108, BD Biosciences). When staining for intracellular proteins (NANOG, OCT4, SOX2), cells were fixed using BD Cytofix™ fixation buffer (BD Biosciences) and incubated for 15 min at room temperature. Following this, cells were permeabilised with 0.1% (v/v) Triton X-100 (Sigma) in BD Pharmigen™ Stain Buffer for 15 min at room temperature before proceeding to staining. Isotype controls were performed for each antibody (SSEA4 isotype, 130-104-608, Myltenyi; SSEA3 isotype, 400811, Biolegend; CD30 isotype, 555749, BD Biosciences; TRA-1-60 isotype, 401618, Biolegend; NANOG isotype, 557702, BD Bioscience; OCT3/4 isotype, 554680, BD Bioscience; SOX2 isotype, 400136, BD Biosciences). Cells were stained with Live/Dead® discrimination dye (L23105, ThermoFischer Scientific) and phenotype analysis of the live single cell population fraction performed by flow cytometry. Isotype staining was considered as a negative control for each analysis and condition.

Karyotype (G-Banding)

Human ES and iPS cells used for G-band karyotype analysis were fixed in suspension. Multiple metaphase spreads were analysed per sample and the number of chromosomes and G-banding pattern were determined.

Differentiation Assays

For spontaneous differentiation, cells cultured in AI media were switched into MEF culture media (10% FBS) for 6 or 12 days. Cells were then fixed for immunofluorescence analysis for the three germ layer lineages.

For directed differentiation, cells adapted to AI media were taken through the hepatocyte differentiation protocol detailed in Hannan et al., 2013, Nat Protoc: doi:10.1038/nprot.2012.153. Adapted H9 cells were dissociated to single cells using Accutase then resuspended in AI media containing ROCK inhibitor (Y27632, Tocris) at 10 μM. Cells were seeded at 1×10⁵/cm² onto gelatinized tissue culture plates pre-coated with MEF media. AI media was replenished 24 hours later. The hepatocyte differentiation protocol was started 48 hours after seeding, at which point cells were transferred to hypoxia conditions (5% O₂, 5% CO₂, 37° C.), which were maintained throughout the differentiation. Cells were fixed for immunofluorescence 3 days (endoderm), 8 days (foregut endoderm) and 25 days (mature hepatocytes) following differentiation induction.

Results

A Chemically Defined Minimal Medium Comprising Activin and IGF1 (AI) is Sufficient to Support Human Pluripotency

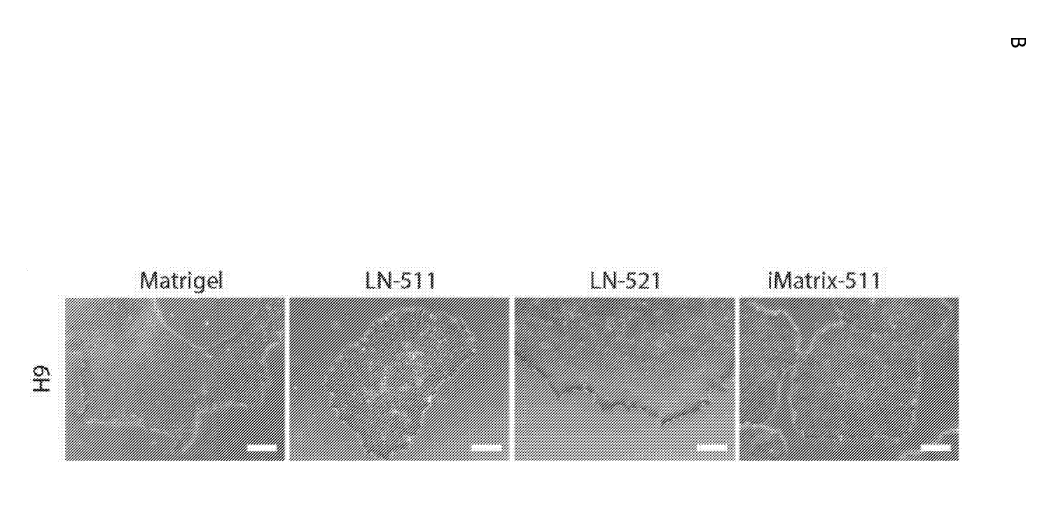

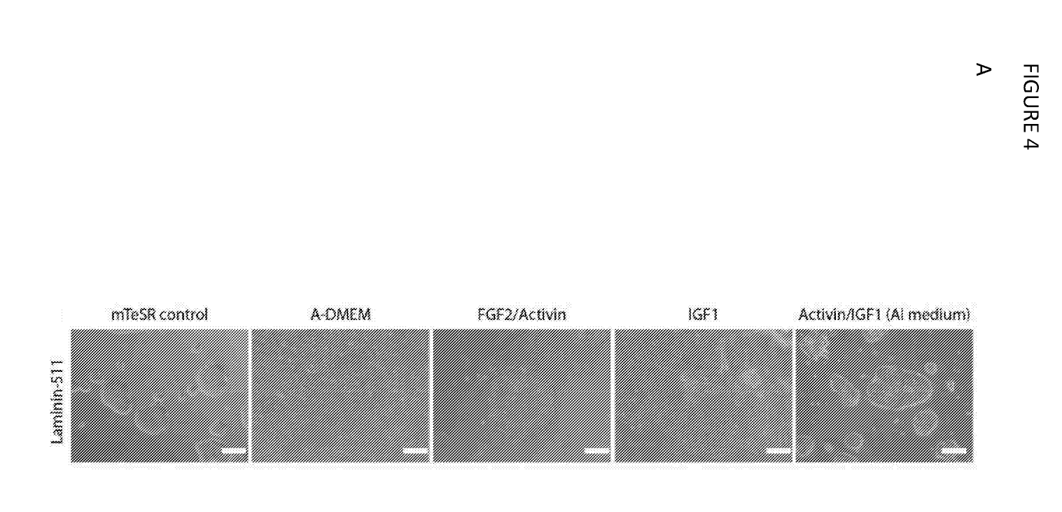

We first used established hES cells to determine whether IGF1 could replace FGF2 in pluripotency culture media. Given the physiological relevance of Laminin-511 we continued with this refined basement membrane component for pluripotent cell culture in chemically defined conditions unless otherwise noted. To test conditions for establishing a viable culture system, H1 hES cells were grown in either control mTeSR™1 media, in basal medium (Advanced-DMEM/F12) with glutamine supplement (2 mM), or basal medium supplemented with glutamine, 12 ng/ml FGF2 and 10 ng/ml Activin (growth factor concentrations from Vallier et al., 2005, J Cell Sci, 118, 4495-4509), glutamine and 17 nM IGF1, or glutamine, 10 ng/ml Activin and 17 nM IGF1 (FIG. 4A). hES cells cultured in Activin and IGF1 (hereafter AI) medium resembled control mTeSR™1 treated cells with a high nuclear to cytoplasmic ratio, tightly packed colonies and distinct colony boundaries.

We next tested methods for passaging the cells in AI medium and found that either manual picking, salt-free PBS, gentle cell dissociation buffer or ReLeSR could be used to propagate the cells, but not EDTA alone (FIG. 4B). We also determined whether the cells in AI medium could be disaggregated to a single-cell level, which is important for the establishment of clonal cell lines, or during the derivation of transgenic or genetically modified hES cells. We confirmed that single cells could be disaggregated with Accutase in the presence of the Rho-associated kinase (ROCK) inhibitor and could be further propagated similar to those passaged as clumps (FIG. 4C).

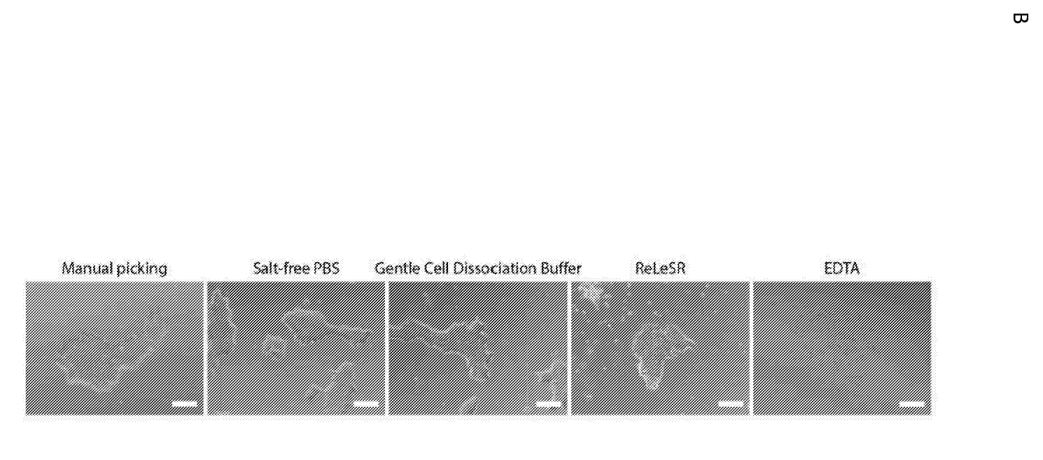

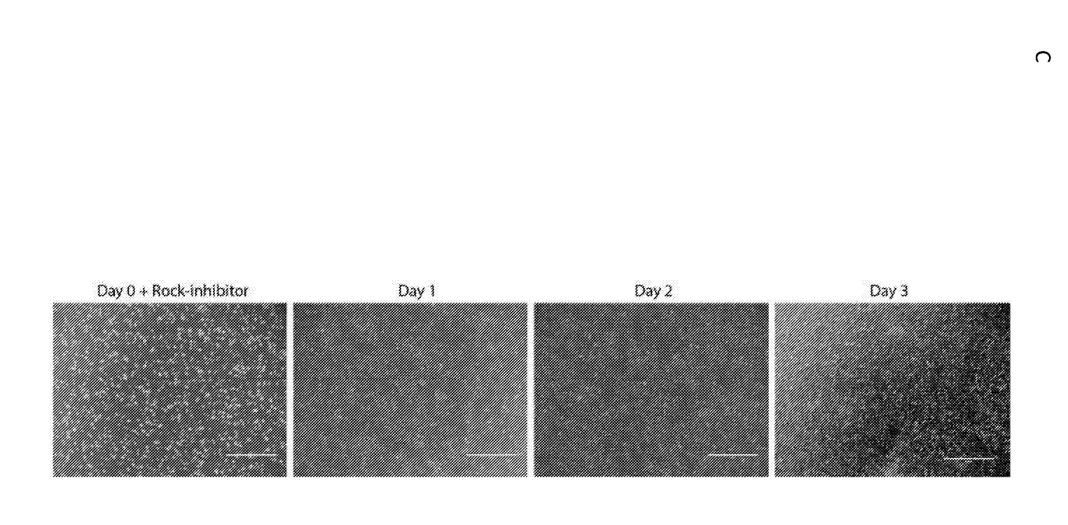

We sought to next investigate whether hES cells cultured in AI medium were maintained independently of FGF signaling. We treated cell cultured in AI medium with inhibitors of FGF receptors or downstream MEK/ERK signaling (FIG. 5A). Cells in AI medium supplemented with an FGF receptor and MEK/ERK inhibitor retained the morphology of pluripotent cells including tightly packed distinct colonies with a high nuclear to cytoplasmic ratio within cells, similar to DMSO-treated control hES cells. By contrast, as expected when we treated cells with an inhibitor of the TGFβ signaling pathway the hES cells underwent apoptosis suggesting the importance of this signaling pathway for hES cell maintenance (FIG. 5A). We confirmed by immunofluorescence analysis that cells cultured in AI medium supplemented with an FGF receptor or MEK/ERK inhibitor retained the expression of the pluripotency proteins OCT4 and NANOG (FIG. 5B). By contrast, in the few colonies that could be observed following 3 days of TGFβ inhibitor treatment, NANOG expression was undetectable in the majority of cells (FIG. 5B). Altogether this shows that while hES cells in AI medium depend on TGFβ signaling for their maintenance, hES cells can be cultured independently of FGF signaling.

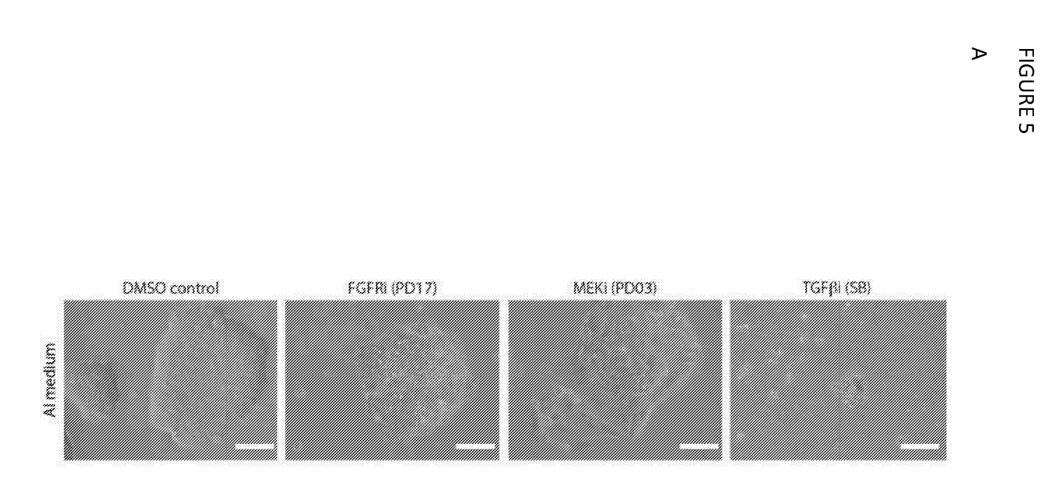

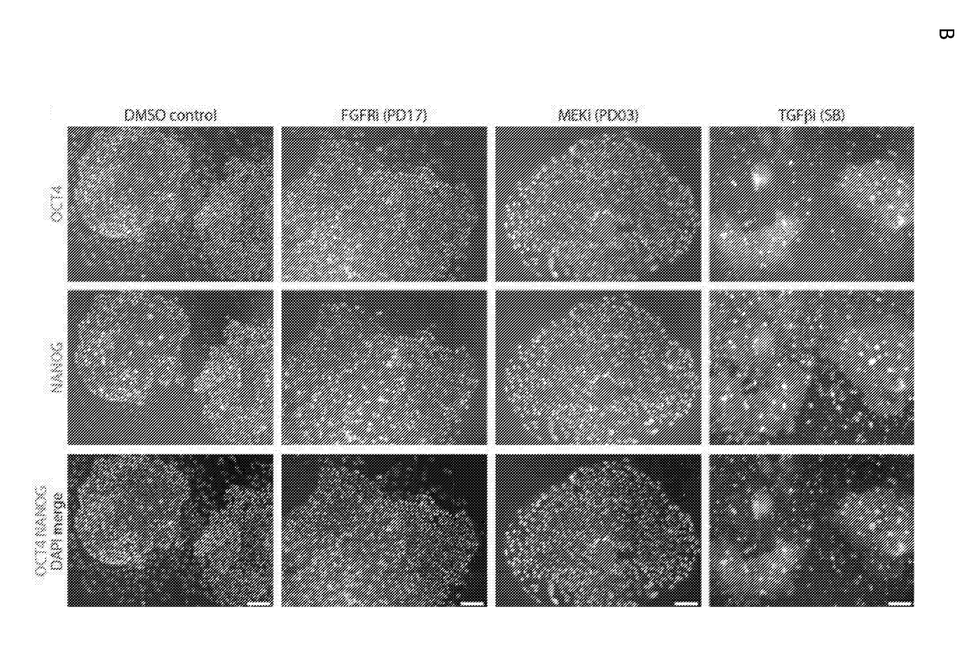

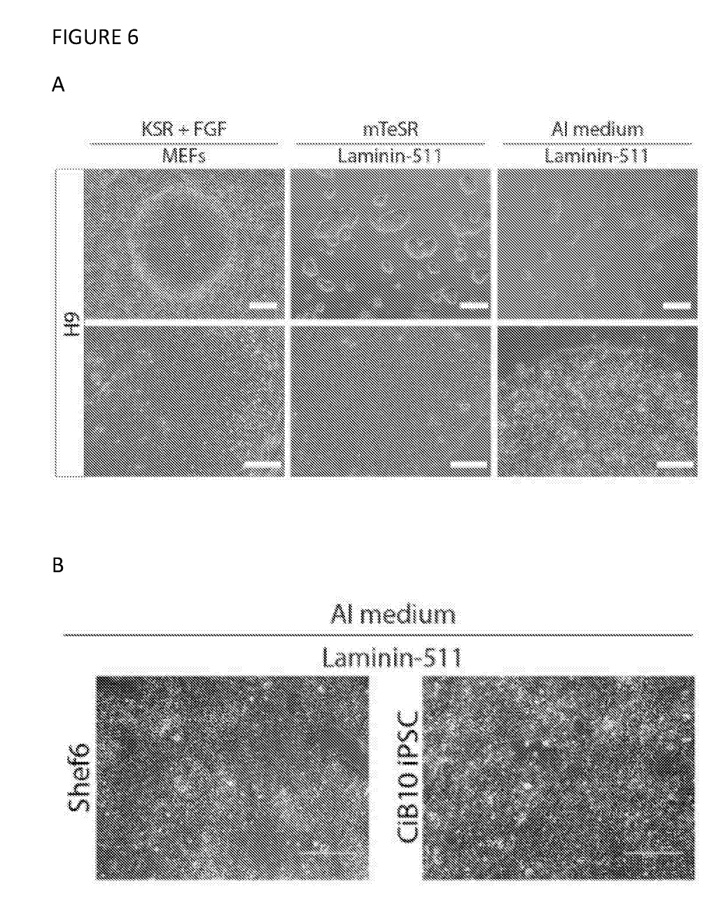

Independent hES cells (H1 and H9) have been propagated in AI medium on Laminin-511 for more than 25 passages over 6 months. Cells were passaged on an approximate 4 to 5 day cycle using ReLeSR and retained their pluripotent morphology, comparable to cells in mTeSR™1, or KSR+FGF (FIG. 6A). Both hES cells (Shef6) and iPS cells (CiB10) cells were also independently propagated in AI medium on Laminin-511 (FIG. 6B).

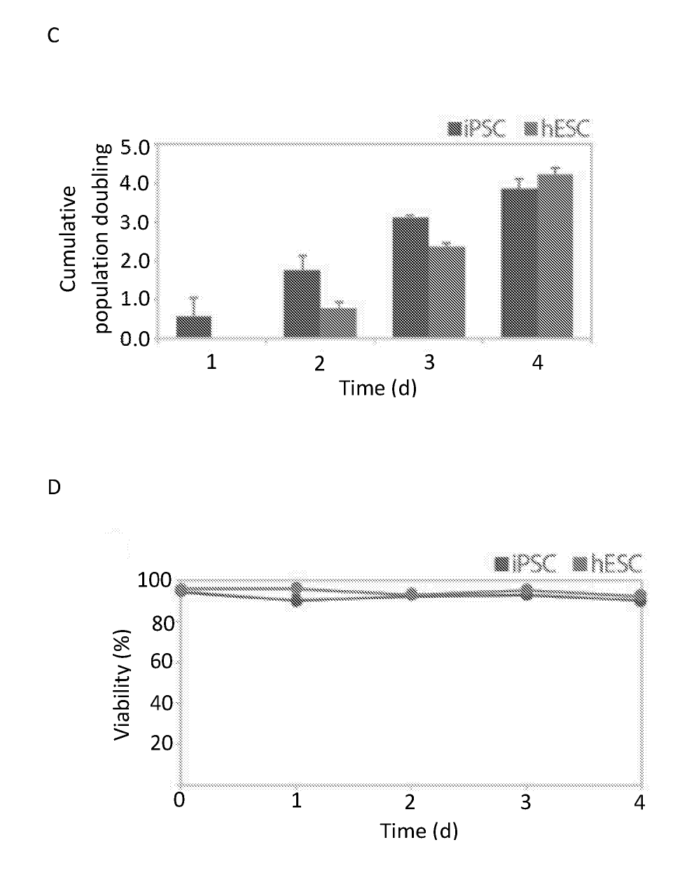

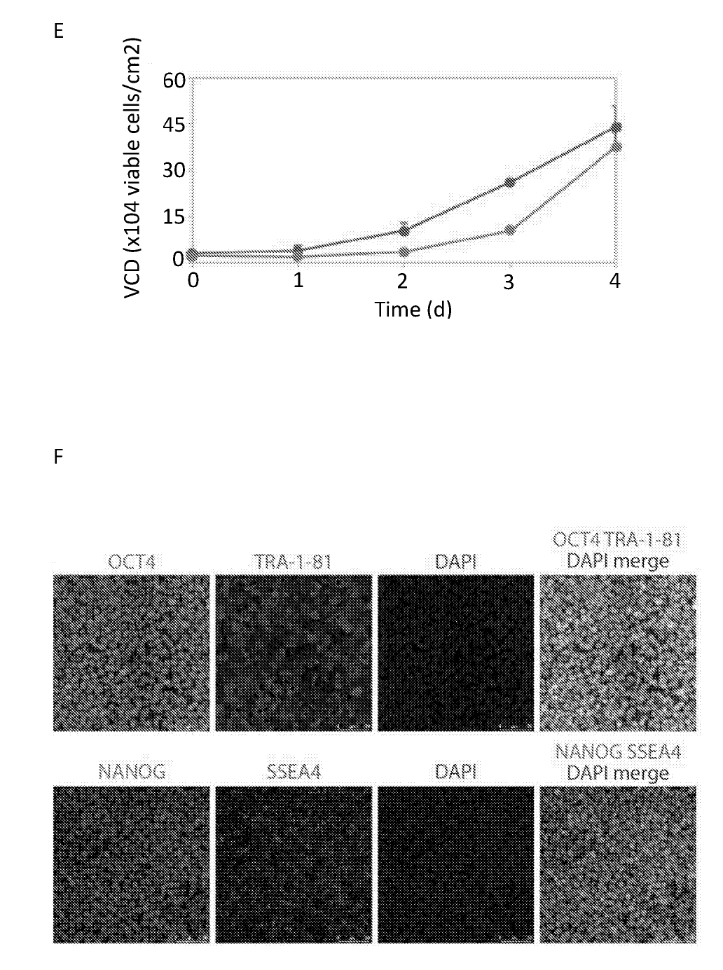

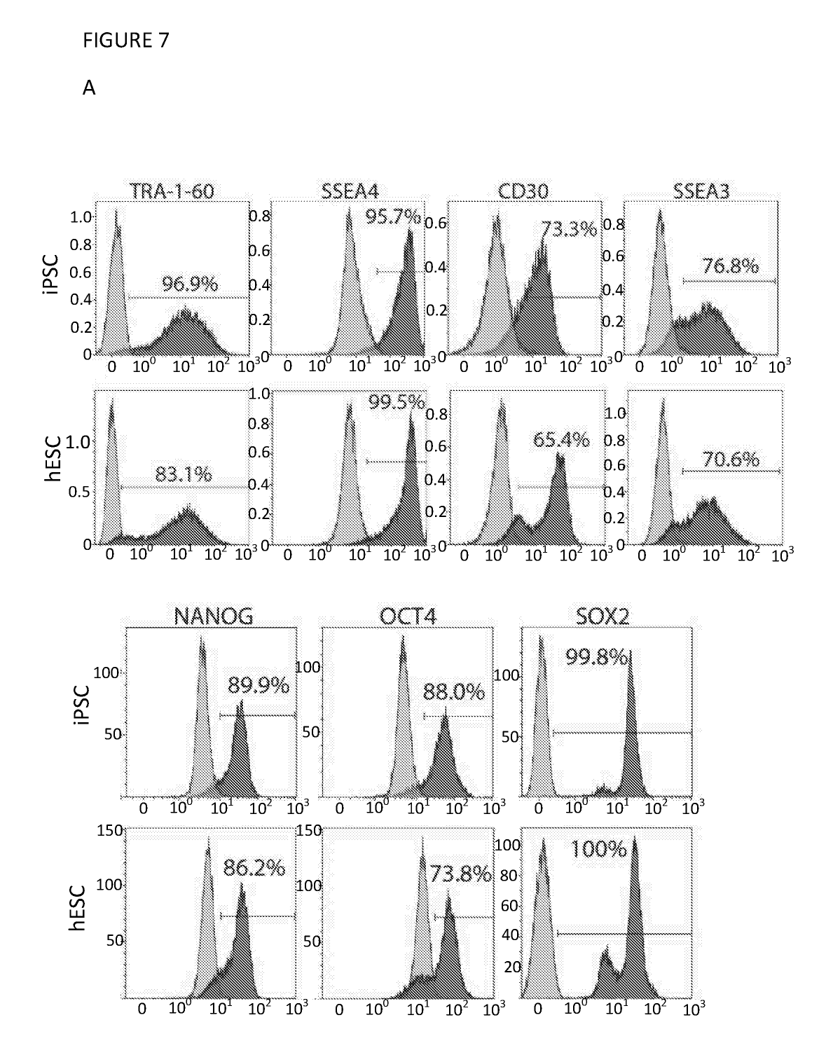

The hES and iPS cells cultured in AI medium were able to double their population every day and retained high viability (FIGS. 6C, 6D and 6E). Human ES and iPS cells cultured in AI medium robustly expressed pluripotency factors OCT4, NANOG, TRA-1-81 and SSEA4, as determined by immunofluorescence analysis (FIG. 6F). Flow cytometry analysis confirmed that a high proportion of the hES and iPS cells expressed the pluripotency-associated cell surface antigens TRA-1-60, SSEA4, CD30 and SSEA3 as well as the nuclear proteins NANOG, OCT4 and SOX2 (FIG. 7A). This was maintained when the cells were propagated using single-cell passaging methods or as clumps of cells over several passages (FIGS. 7B and 7C). Moreover, G-band karyotype analysis confirmed that both hES and iPS cells retained a normal complement of 46 chromosomes (FIG. 7D).

AI Medium Supports the Derivation of hES Cells from Human Embryos, and iPS Cell Reprogramming from Fibroblasts

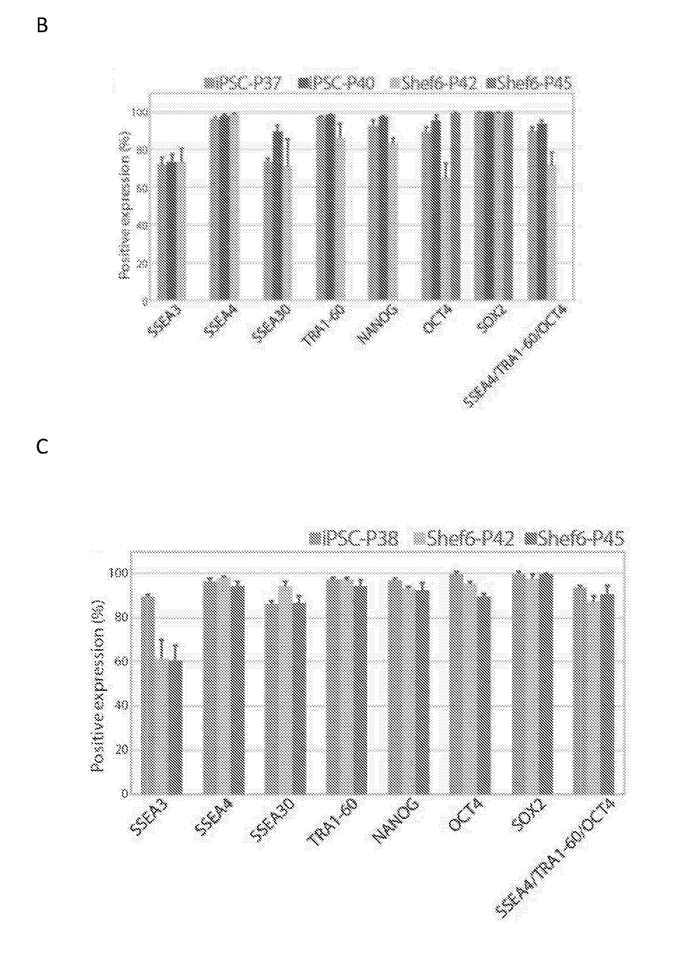

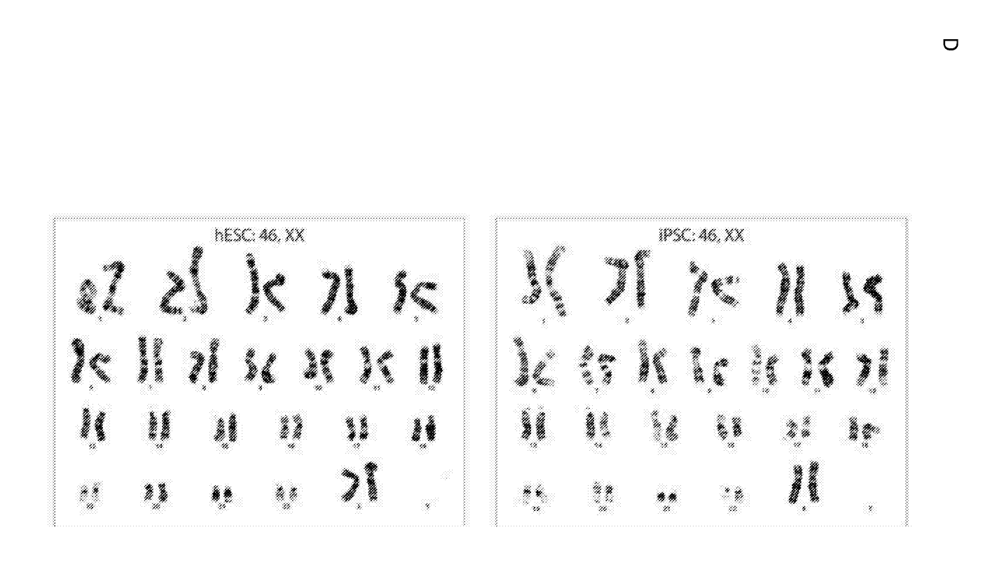

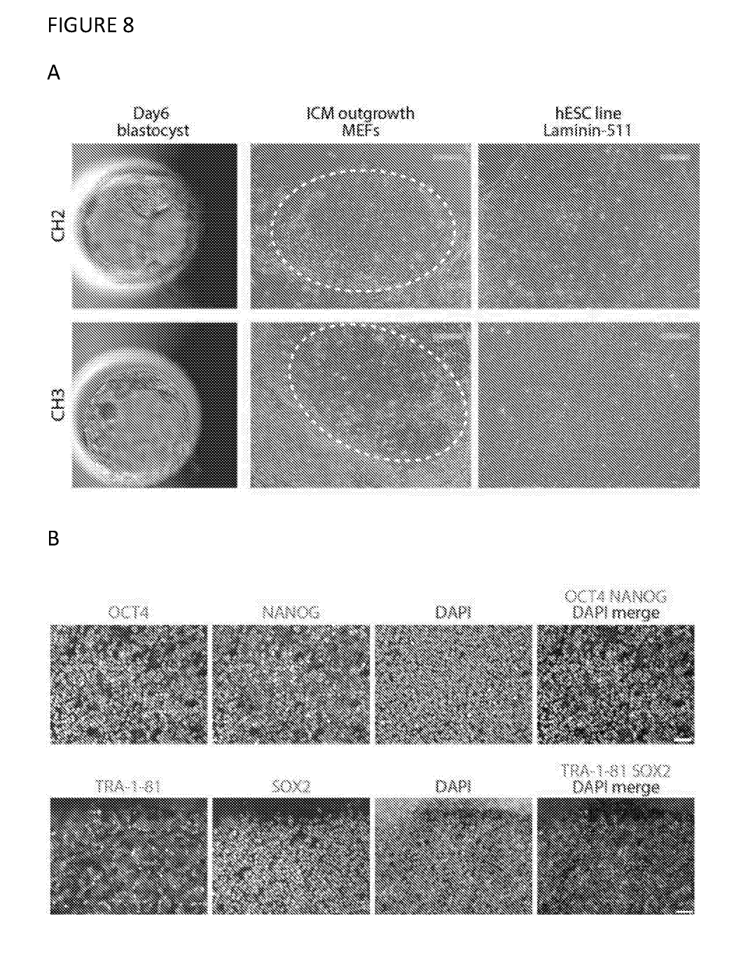

As a stringent test of these culture conditions, we next asked whether AI medium could be used to derive hES cells directly from human embryos. Day 5 blastocysts were cultured overnight to day 6 in human embryo culture media. The inner cell mass (ICM) and overlaying polar trophectoderm were microdissected from the mural trophectoderm and the ICM clump was plated in AI medium (FIG. 8A). Human ES cell-like colonies emerged within the first week after plating in AI medium and could be maintained as stable hES cell lines on Laminin-511 (FIG. 8A). Immunofluorescence analysis confirmed that hES cells expressed pluripotency proteins NANOG, OCT4 SOX2 and TRA-1-81 (FIG. 8B).

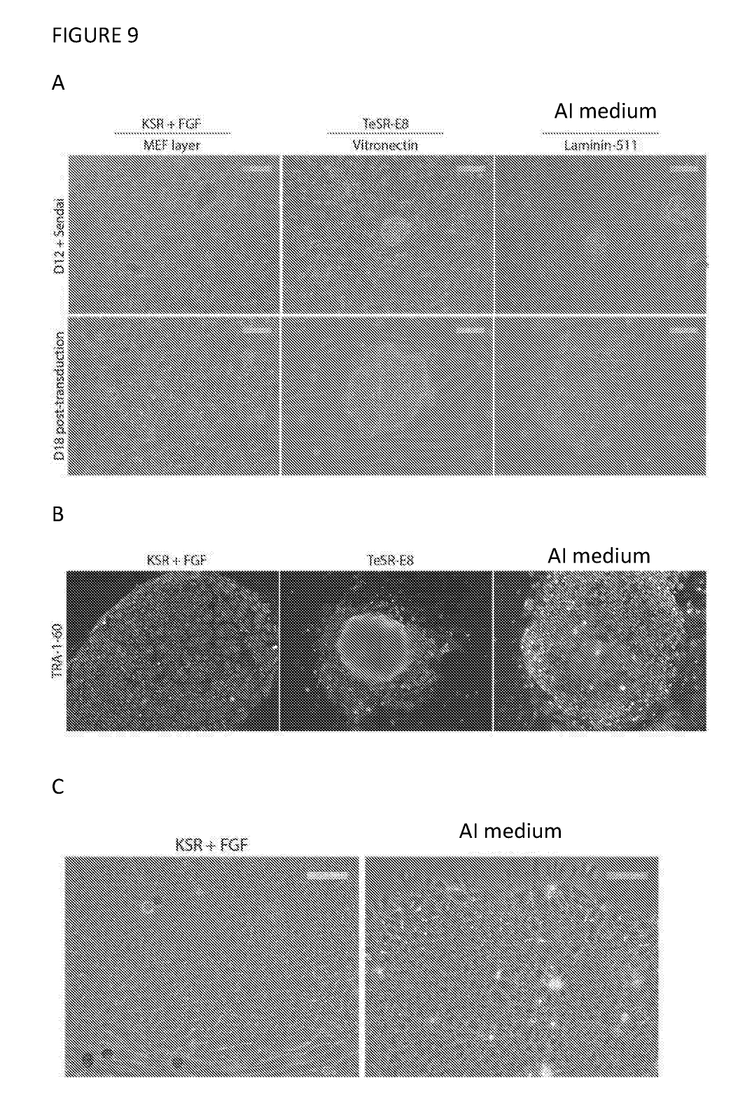

We also sought to determine if AI medium could be used to support the derivation of iPS cells from differentiated fibroblasts using standard reprogramming methods. We virally transduced BJ fibroblasts with Sendai virus vectors harboring the reprogramming factors OCT4, SOX2, KLF4 and c-MYC. Following viral transduction the fibroblast cells were placed into AI medium, KSR+FGF, or TeSR™-E8 medium. Colonies resembling pluripotent stem cells emerged within 12 days following infection (FIG. 9A, circles). The emerging colonies were live cell stained for TRA-1-60 which confirmed their iPS cell-like identity (FIG. 9B). Colonies established in AI medium continued to expand over time and were passaged with Accutase to establish stable iPS cell lines (FIG. 9C). Altogether this demonstrated that AI medium was sufficient to allow for the establishment of self-renewing iPS cell colonies.

Spontaneous and Directed Differentiation of hES Cells Cultured in AI Medium

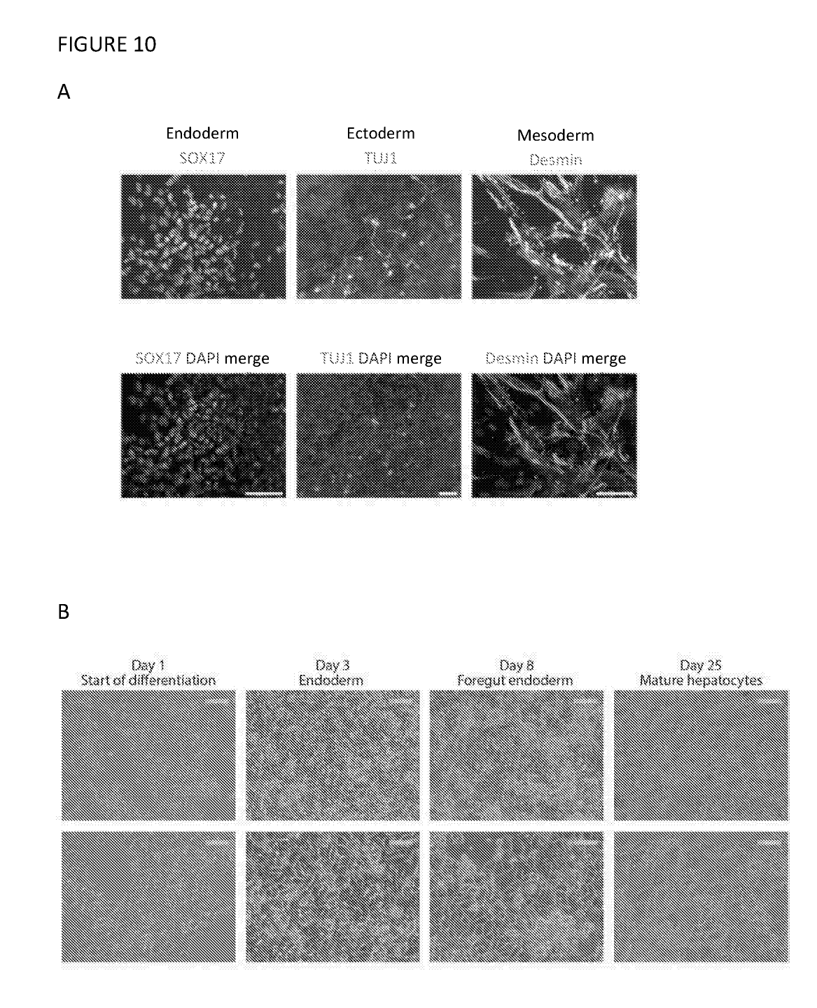

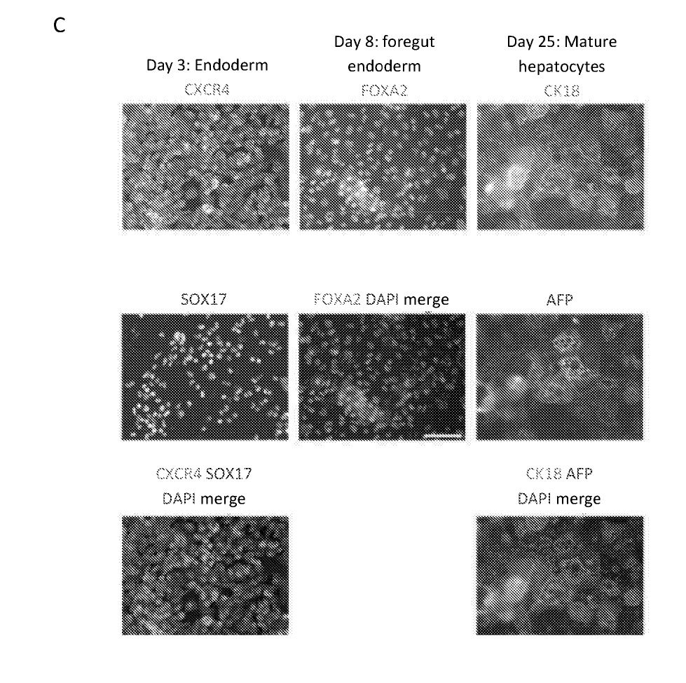

We confirmed that hES cells derived exclusively in AI medium were pluripotent and retained the capacity for germ layer differentiation. Immunofluorescence analysis of spontaneously differentiating hES cells demonstrated their ability to differentiate into SOX17-expressing endoderm cells, TUJ1-expressing ectodermally-derived neurons and Desmin-expressing mesoderm cells (FIG. 10A).

We next tested the ability of hES cells adapted to AI medium to differentiate into functional hepatocytes, using a standard protocol (Hannan et al., 2013, Nat Protoc. 8, 430-437). Hepatocyte differentiation is of clinical relevance not only to understand the etiology of liver-associated disease, but also to perform large-scale screens for drug toxicity prior to clinical trials and to potentially generate cells for cell replacement therapies.

We observed obvious changes in cell morphology during the hepatocyte differentiation process (FIG. 10B), consistent with what has been previously reported. Immunofluorescence analysis confirmed the identity of the initial pan-endoderm cells that express SOX17 and CXCR4, followed by foregut endoderm cells that express FOXA2, and eventual mature hepatocytes that express AFP and Cytokeratin-18 (FIG. 10C).

All documents referred to herein are hereby incorporated by reference in their entirety, with special attention to the subject matter for which they are referred. Various modifications and variations of the described methods and system of the invention will be apparent to those skilled in the art without departing from the scope and spirit of the invention.

Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology, cellular immunology or related fields are intended to be within the scope of the following claims.

1. A cell medium for culturing a pluripotent stem cell, wherein said medium is a chemically defined or minimal medium which comprises insulin-like growth factor 1 (IGF1) and which is free or substantially free from fibroblast growth factor (FGF) and activators of any FGF receptor, and which does not comprise an ErbB3 ligand.

2. The cell medium of claim 1 for culturing an embryonic stem cell or induced pluripotent cell.

3. The cell medium of either of claim 1 or 2 for culturing a mammalian cell.

4. The cell medium of claim 3 for culturing a human cell.

5. The medium of any one of claims 1 to 4 wherein said IGF1 comprises the sequence of SEQ ID NO:1 or SEQ ID NO:2.

6. The medium of any one of claims 1 to 5 further comprising a TGF-β family member.

7. The medium of claim 6 wherein said TGF-β family member is Activin.

8. A cell medium for culturing a human pluripotent stem cell, wherein said medium is a chemically defined or minimal medium and wherein said medium consists of a base medium, IGF1, a TGF-β family member and a glutamine supplement.

9. The medium of claim 8 wherein said TGF-β family member is Activin.

10. The medium of claim 8 or 9 wherein said base medium is advanced DMEM/F12.

11. An in vitro method for culturing a pluripotent stem cell, said method comprising culturing said cell in the medium according to any one of claims 1 to 10.

12. Use of the medium of any one of claims 1 to 10 in the culture of a pluripotent stem cell.

13. The method of claim 11 or the use of claim 12 wherein said cell is an embryonic stem cell or an induced pluripotent cell.

14. The method or use of claim 13 wherein said cell is a mammalian cell.

15. The method or use of claim 14 wherein said cell is a human cell.

16. A cell obtainable or obtained by the method of any one of claims 11 to 15.

17. A medium for culturing an embryo, wherein said medium is as defined in any one of claims 1 to 10.

18. An in vitro method for culturing an embryo, comprising culturing said embryo in the medium of claim 17.

19. The method of claim 18 wherein said embryo is a human embryo.

20. Use of the medium of claim 17 in the culture of an embryo.

21. The method or use of any one of claims 11 to 15 or 17 to 20 wherein said culture is carried out in conjunction with a basement membrane.

22. The method or use of claim 21 wherein said basement membrane comprises laminin.

23. A method of screening for factors that are essential for the culture of human embryos, wherein said method comprises the following steps:
(i) Preparing a medium by adding a factor to be analysed;
(ii) Culturing a human embryo in said medium;
(iii) Comparing said embryo to an embryo that has been cultured in a medium without said factor or in the presence of an activator or inhibitor of said factor; and
(iv) Determining whether said factor is essential for culture of said embryo.
